SEQUENCE LISTING (9 sequences)

SEQ ID NO: 1 - protein, 241 aa, Homo sapien
MSMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 247 aa, Homo sapien
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 3 - protein, 257 aa, Homo sapien
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 4 - protein, 210 aa, Homo sapien
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 5 - protein, 5 aa, Artificial Sequence
  Synthetic sequence
XXRVX

SEQ ID NO: 6 - protein, 6 aa, Artificial Sequence
  Synthetic Sequence
XXRVXX

SEQ ID NO: 7 - protein, 7 aa, Artificial Sequence
  Synthetic Sequence
XXRVXXX

SEQ ID NO: 8 - protein, 8 aa, Artificial Sequence
  Synthetic Sequence
XXRVXXSX

SEQ ID NO: 9 - protein, 1159 aa, Homo sapiens
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQGNHSGVVLSINSREMHSYLVS

(19) United States
(12) Patent Application Publication
(10) Pub. No.: US 2013/0336988 A1
(43) Pub. Date: Dec. 19, 2013

(54) METHODS FOR TREATING EARLY STAGE OR MILD NEUROLOGICAL DISORDERS

(71) Applicants: Barbara L. Hempstead, New York, NY (US); Taeho Kim, Palo Alto, CA (US); Katrin Deinhardt, Southampton (GB); Moses Victor Chao, New York, NY (US); Jianmin Yang, Forest Hills, NY (US)

(72) Inventors: Barbara L. Hempstead, New York, NY (US); Taeho Kim, Palo Alto, CA (US); Katrin Deinhardt, Southampton (GB); Moses Victor Chao, New York, NY (US); Jianmin Yang, Forest Hills, NY (US)

(73) Assignees: NEW YORK UNIVERSITY, New York, NY (US); CORNELL UNIVERSITY, Ithaca, NY (US)

(21) Appl. No.: 13/988,193

(22) PCT Filed: Nov. 17, 2011
(86) PCT No.: PCT/US11/61125

§ 371 Date: Aug. 30, 2013

Related U.S. Application Data
(60) Provisional application No. 61/414,700, filed on Nov. 17, 2010.

(51) Int. Cl. A61K 39/395; A61K 38/08; A61K 31/713; A61K 31/7088

(52) U.S. Cl. CPC A61K 39/3955; A61K 31/7088; A61K 38/08; A61K 31/713
USPC 424/158.1; 424/172.1; 514/44 R; 514/17.7; 514/44 A

(57) ABSTRACT

This disclosure relates to modulation of the interactions between proNTs and p75^{NTR}/SorCS2 expressed on neuronal cells. Inhibition of such interactions is useful for reducing unwanted synaptic elimination, neurite pruning and/or other neuronal structural collapses, and for treating early stage and mild neurological disorders including mild cognitive impairment.

### CROSS REFERENCE TO RELATED APPLICATION

This application claims the benefit of priority of U.S. Provisional Application No. 61/414,700, filed Nov. 17, 2010, the entire contents of which are incorporated herein by reference.

### STATEMENT REGARDING FEDERALLY SPONSORED RESEARCH OR DEVELOPMENT

This invention was made with Government Support under Contract No. NS30687 and NS64114. The Government has certain rights in this invention.

### FIELD OF THE DISCLOSURE

This disclosure relates to modulation of the interactions between proNTs and p75^{NTR}/SorCS2 expressed on neuronal cells. Such modulation is useful for reducing unwanted neurite pruning and other neuronal structural collapses, and for treating early stage and/or mild neurological disorders including mild cognitive impairment.

### BACKGROUND ART

The development of the nervous system is characterized by the growth and retraction of neuronal processes, which is precisely regulated by many extrinsic factors and signals (KANTOR, D. B. et al., Neuron 38:849-852 (2003); LUO, L. et al., Annu Rev Neurosci 28:127-156 (2005)). The branching and refinement of axons and dendrites must be influenced by mechanisms that also restrict the size and extent of growth. How the balance between growth and retraction is achieved by extrinsic signals is not well understood. In addition to ephrins, neuropilins and semaphorins (FAULKNER, R. L. et al., Dev Neurosci 29:6-13 (2007); XU, N. J. et al., Nat Neuroscience 12:268-276 (2009)), the branching of axons and dendrites is highly influenced by neurotrophins (COHEN-CORY, S. et al., Dev Neurobiol 70: 271-288 (2010); MCALLISTER, A. K. et al., Neuron 15:791-803 (1995)). Neurotrophins, which include nerve growth factor (NGF), brain-derived growth factor (BDNF), NT-3 and NT4, are required for neuronal survival, differentiation, synapse formation and synaptic plasticity (SNIDER, W. D., Cell 77:627-638 (1994)). These proteins are secreted and exert their cellular effects through the actions of two different receptors, the tropomyosin-related kinase (Trk) receptor tyrosine kinase, which transduces the survival and differentiation signals, and the p75 neurotrophin receptor (p75^{NTR}). Neurotrophins are initially synthesized as precursor proteins, or proneurotrophins (proNTs), that are cleaved to produce mature forms of 12-14,000 molecular weight. ProNT processing occurs intracellularly through furin or prohormoneconvertases, or extracellularly through plasmin or matrix metalloproteases (LEE, R. et al., Science 294:1945-1948 (2001); SEIDAH, N. G. et al., FEBS Lett 379:247-250 (1996); SEIDAH, N. G. et al., Biochem J 314(Pt 3):951-960 (1996); SUTER, U. et al., Embo J 10:2395-2400 (1991)). Though much attention has been directed to mature neurotrophins, their precursors are also biologically active. In several cases, proNTs display opposite biological effects to their mature versions, by producing pro-apoptotic effects, as well as long-term depression at synapses (LEE, R. et al., Science 294:1945-1948 (2001); TENG, H. K. et al., Journal of Neuroscience 25:5455-5463 (2005); WOO, N. H. et al., Nat Neuroscience 8:1069-1077 (2005); YANO, H. et al., Journal of Neuroscience 29:14790-14802 (2009)). ProNT-induced cell death has been observed for proNGF, proBDNF and proNT-3 in in vitro systems of peripheral neurons, and the proapoptotic action of proNGF has been described in vivo in numerous injury response paradigms in adult animals, including corticospinal axotomy, spinal cord injury, and acute seizures (HEMPSTEAD, B. L., Neurotox Res 16:255-260 (2009)). ProNTs bind preferentially to a receptor complex of p75^{NTR} and sortilin, a member of the Vps10p domain-containing transmembrane receptors, to mediate cell death (NYKJAER, A., Nature 427:843-848 (2004); TENG, H. K. et al., Journal of Neuroscience 25:5455-5463 (2005)). Mechanistically, the coordinate induction of both proNGF and p75^{NTR} to mediate neuronal death in vivo has been confirmed using both function-blocking antibodies as well as p75^{NTR} null animals to ameliorate cell loss (BEATTIE, M. S. et al., Neuron 36:375-386 (2002); HARRINGTON, A. W. et al., Proc Natl Acad Sci USA 101:6226-6230 (2004); OLOSIN, M. et al., Journal of Neuroscience 28:9870-9879 (2008)). Downstream of the p75^{NTR}/sortilin complex, activation of JNK and cleavage of caspase-3 transduce an apoptotic signal (KOSHIMIZU, H. et al., Neurosci Lett 473:229-232 (2010); YANO, H. et al., Journal of Neuroscience 29:14790-14802 (2009)).

In addition to the proapoptotic properties of proNTs, alternative actions for proBDNF at the synapse were recently identified, including promotion of long-term depression upon delivery of recombinant protein, and the requirement for processing of endogenous proBDNF by extracellular proteases to induce long-term potentiation (PANG, P. T. et al., Science 306:487-491 (2004); WOO, N. H. et al., Nat Neuroscience 8:1069-1077 (2005)). Together, this suggests a general bidirectional mechanism of proNT/neurotrophin function beyond cell death and survival decisions. However, the molecular mechanism downstream of proNTs that underlies the differential outcomes is still poorly characterized.

### SUMMARY OF THE DISCLOSURE

This disclosure is directed to methods for inhibiting the interactions between proneurotrophin (proNT) and p75^{NTR}/SorCS2 expressed on neuronal cells. Such inhibition is desirable for controlling, reducing and/or preventing unwanted reduction in synaptic spines, neuronal growth cone collapse, and/or neurite pruning.

Inhibition of the interactions between proNT and p75^{NTR}/SorCS2 expressed on neuronal cells can be achieved through administration of an antagonist which interferes with such interactions. The antagonist can be a proNT antagonist which includes a neutralizing antibody that binds specifically to a proNT, an aptamer, an oligopeptide, a small molecule compound, or a nucleic acid molecule which reduces the level or activity of a proNT mRNA, for example. Alternatively, the antagonist can be a SorCS2 antagonist which includes an anti-SorCS2 antibody, an aptamer, an oligopeptide, a small molecule compound, or a nucleic acid molecule which reduces the level or activity of the SorCS2 mRNA. The antagonist can also be a p75^{NTR} antagonist, which includes a neutralizing antibody that binds specifically to a p75^{NTR}, an aptamer, an oligopeptide, a small molecule compound, or a nucleic acid molecule which reduces the level or activity of a p75^{NTR} mRNA.

The methods disclosed herein can be used for treating mild or early stage neurological disorders, including mild cognitive impairment, early stage neurodegenerative disorders, and other disorders which involve loss of structure or function of neurons at a relatively early stage.

### BRIEF DESCRIPTION OF THE DRAWINGS

The patent or application file contains drawings executed in color. Copies of this patent or patent application publication with color drawings will be provided by the Office upon request and payment of the necessary fee.

FIG. 1A-F. proBDNF induces morphological defects in vivo. (A) Strategy for the generation of the probdnf-HA knock-in mouse. (B, C) Sholl analysis of dentate granule neurons from P30 (B) or P105 (C) mice. 40 neurons from 4-5 animals were traced per genotype. All results are presented as mean+/−SEM. The differences between +/+ and probdnf-HA/+ mice, and the differences between bdnf^{+/-} and probdnf-HA/+ mice were significant (* p<0.01, two-way ANOVA). (D) Representative traces of Golgi-stained dentate granule neurons from bdnf wild type (+/+*), bdnf haploinsufficient mice (bdnf^{+/-}), wild type littermates of probdnf-HA/+(+/+) and pro-bdnf-HA/+ mice. (E) Expression of proBDNF led to a loss of synaptic spine density in two classes of hippocampal neurons. (F) Reduced hippocampal volume in probdnf-HA knock-in mice. Total hippocampal volume was measured from MRI images of 11 months old bdnf wild type (+/+*, n=3), BDNF heterozygous mice (bdnf^{+/-}, n=5), bdnf-HA knock-in (bdnf-HA/+, n=4) and probdnf knock-in (probdnf-HA/+, n=5) mice. The results are shown as mean+/−SEM (**p<0.01, n.s. p=0.12; Student's t-test).

FIG. 2A-G. ProNTs induce growth cone collapse. (A, B) DIV3 hippocampal neurons transfected with LifeAct-RFP were imaged by time-lapse microscopy before (top panel) and starting approximately 2 min after (bottom panel) treatment with proBDNF (A) or proNGF (B). Actin dynamics stalled upon proNT treatment and subsequently the growth cone collapsed. Time is indicated in (min:sec). Scale bars, 10 μm. (C, D) DIV3 hippocampal cultures were fixed and stained for p75^{NTR} (C) and SorCS2 (D). Arrows highlight growth cones. Scale bars, 20 μm. (E) Brain sections from 6-week old C57/BL6 mice were stained for SorCS2. DG, dentate gyrus. Arrows highlight expression in dentate granule dendrites. Scale bar, 200 μm. (F) Both sortilin and SorCS2 interact with proBDNF. HEK 293 cells were transfected with proBDNF-HA and myc-sortilin or mycSorCS2, lysates were immunoprecipitated with anti-HA agarose and analyzed by Western blot with indicated antibodies. (G) IP-WB from 293T cells showing interactions between proNGF and sortilin and SorCS2. Note that proNGF co-immunoprecipitated with both sortilin and SorCS2.

FIG. 3A-D. p75^{NTR} and SorCS2 are required for growth cone collapse. (A) E15 DIV3 mouse hippocampal neurons were treated with proNGF or K252a and proBDNF for 20 min, fixed and stained for actin, fascin and p75^{NTR}. Incubation with either proNT led to collapse of growth cones in p75-positive cells (arrows), while p75-negative cells were not affected (*). Scale bars, 10 μm. (B) Quantification of the data in (A). Ctl, untreated controls; proNGF, cells exposed to 10 ng/ml proNGF; ctl*, cells treated with K252a and control medium; proBDNF, cells incubated with K252a and medium containing 10 ng/ml proBDNF; NGF, cells incubated with 5 ng/ml NGF. ***, p<0.001; n.s., not significant; one way ANOVA followed by Tukey's T-test. (C) Quantification of the data in (D). ***, p<0.001; n.s., not significant; one way ANOVA followed by Tukey's T-test. (D) Ectodomain antibodies against SorCS2 prevented proNGF-induced retraction. Cells were preincubated with anti-SorCS2 antibodies prior to proNGF addition. Neurons were fixed and stained for actin, fascin and p75^{NTR}. Scale bars, 10 μm.

FIG. 4A-H. Trio displacement from the p75^{NTR}/SorCS2 complex leads to growth cone collapse. (A) Co-immunoprecipitation of endogenous Trio with p75^{NTR} from HT1080 cells expressing both p75 and sortilin or SorCS2, but not p75 alone. (B, C) The Trio kinase domain interacts with p75^{NTR}. Myc-Trio domains were co-expressed with HA-p75^{NTR} in 293T cells, immunoprecipitated with anti-Myc (B) or with anti-HA (C) antibodies, and Western blots were probed with the indicated antibodies to detect coimmunoprecipitation. (D) Schematic representation of full-length Trio. (E, F) proNT treatment leads to a dissociation of the p75^{NTR}/Trio/SorCS2 complex. (E) HT1080 cells expression both p75^{NTR} and SorCS2 were incubated with proNGF or proBDNF for 20 min, cells were lysed and p75^{NTR} was immunoprecipitated. Western blots were probed with indicated antibodies to detect co-precipitation. (F) Quantification of (E). Shown is the mean+SEM from at least four independent experiments. ***, p<0.001; n.s., not significant; one way ANOVA followed by Tukey's T-test. (G) Endogenous Trio localized to actin-rich structures in hippocampal neurons. Hippocampal neurons DIV3 were fixed and stained for indicated proteins. (H) Expression of the Trio kinase domain induced collapse. Hippocampal neurons were transfected with the Trio kinase domain at DIV2, fixed at DIV3 and stained with indicated antibodies. Scale bars, 10 μm.

FIG. 5A-F. proNT stimulation leads to decreased Rac activity. (A) DIV2 cortical neurons were with proNGF or K252a (control*) in presence or absence of proBDNF for 20 min and lysed. Lysates were incubated with GST-PAK-CRIB beads to isolate activated Rac. As a control, lysates were incubated with GDP or GTPγS for 30 min prior to the pull down. (B) Isolated activated Rac was measured by densitometry and normalized to the input. Shown is the mean+SEM from at least four independent experiments. ***, p<0.001 one way ANOVA followed by Tukey's T-test. (C) Treatment with the Rac inhibitor EHT 1864 or the Trio GEF1 inhibitor ITX3 leads to decreased Rac activity in primary neuronal cultures. Active Rac was isolated as described above. (D) Inhibition of Rac activity or Trio GEF1 activity induce growth cone collapse. DIV3 hippocampal neurons were treated with EHT 1864 or ITX3, fixed and stained for actin. Arrows indicate collapsed growth cones. (E) Expression of the Trio GEF1 domain rescues proNGF induced retraction. Hippocampal neurons were transfected with the Trio GEF1 domain at DIV2. The following day, cells were treated with proNGF, fixed and stained with indicated antibodies. (F) Quantitation of (E). Shown is the mean+SEM of four independent experiments; n.s., not significant, Student's t-test. Scale bars, 10 μm.

FIG. 6A-D. PKC-dependent fascin inactivation contributes to growth cone collapse. (A) Hippocampal neurons were pretreated with the PKC inhibitor Gö6976, followed by addition of proNGF. While the inhibitor alone had no effect on actin morphology, proNGF failed to induce full collapse in cells pretreated with Gö6976. (B) Quantification of proNGF-induced collapse in cells pretreated with Gö6976 or the small inhibitory peptide 20-28; n.s., not significant, Student's t-test. (C, D) Expression of constitutively active, phosphorylation-deficient fascin^{SER(36, 38, 39)ALA} prevents collapse of the growth cone upon proNGF treatment. Cells were transfected with fascin^{SER(36, 38, 39)ALA}. 24 h later neurons were treated with proNGF, fixed and stained for fascin, actin and p75^{NTR}. (C) Quantitation of the data in (D); n.s., not significant, Student's t-test. Scale bars, 10 μm.

FIG. 7. Model for acute proNT action on actin dynamics. The p75^{NTR}/SorCS2 receptor complex is associated with the Rac GEF Trio and therefore localizes Rac activity to dynamically expanding growth cone structures. Upon proNT binding, Trio dissociates from the complex and Rac activity decreases. Subsequently, filopodial formation is abolished. In parallel, PKC is activated and phosphorylates, and therefore inactivates the actin bundling protein fascin. This leads to a destabilization of existing actin filaments and as a consequence, their collapse.

### DETAILED DESCRIPTION

The present inventors have discovered that proNTs can exert acute actions on neuronal cell shape by rapidly inducing growth cone collapse. These actions require a co-receptor for p75^{NTR}, the sortilin family member SorCS2. The inventors have also discovered that downstream of proNTs, the coordinate activation of two separate signaling pathways results in the collapse of actin filaments and subsequent process retraction. First, the actin bundling protein fascin is inactivated through phosphorylation upon proNT binding, leading to destabilization of actin filaments. Second, displacement of the activating protein Trio from actin-rich protrusions leads to decreased activity of the small GTPase Rac, and reduction of filopodia formation. The synchronized deactivation of these two pathways ultimately leads to growth cone retraction, an initial step in neurite pruning. The inventors have further demonstrated that this proNT-induced pruning of processes and reduction in the number of synaptic spines occurs in vivo during neuronal development. Consistent with these findings, this disclosure provides methods of inhibiting the interactions between proNTs and p75^{NTR}/SorCS2 expressed on neuronal cells. Such inhibition is desirable for controlling, reducing and/or preventing unwanted neuronal growth cone collapse or reduction in synaptic spines and/or neurite pruning, and is useful for treating mild or early stage neurological disorders.

Neurological Disorders to be Treated

The methods disclosed herein are particularly useful for treating neurological disorders which involve loss of structure or function of neurons at a relatively early stage or which exhibit only mild symptoms. Such early stage or mild disorders may be characterized at the cellular level by neurons that have begun to lose their processes (neuritis), or have begun to show a reduction in synaptic spines, without being apoptotic or necrotic. The term “neuronal processes” includes both types of protrusions from the cell body of a neuron: axons and dendrites. The dendritic field of a neuron is filled by both dendrite and dendritic spines (or synaptic spines), and hence the dendritic complexity is affected by both dendrites and synaptic spines. In some embodiments, an early stage or mild neurological disorder may be associated with a significant reduction of synaptic spines only, without a significant reduction in dendrites or axons. In other embodiments, the disorder may be associated with a significant reduction of both synaptic spines and dendrites, and optionally additionally with a reduction of axons. Dendritic complexity can be measured and determined using established techniques, e.g., imaging techniques including cellular or molecular imaging, or structural or functional magnetic resonance imaging. Reductions in dendritic complexity can be manifested by reductions in hippocampal volume (see, e.g., Chen et al., Science 314(5796):140-3 (2006)). A reduction can be determined in comparison to a control (either a normal subject or the same test subject at an earlier, healthy time). A reduction is considered to be “significant” if the extent of reduction is apparent upon visual examination of images, or is, when quantified, beyond experimental margin of error, e.g., at least 10%, 20%, 30%, 40%, 50% or more. The disorders may have clinical symptoms such as certain mild to moderate cognitive or movement dysfunction. Examples of such disorders include mild cognitive impairment, early stage or mild neurodegenerative disorders, and other disorders that are associated with mild neurological dysfunction.

“Mild cognitive impairment” (MCI) is well defined clinically. See, e.g., Petersen, N Engl J Med 364 (23): 2227-2234 (2011); Snyder et al., Alzhermer's & Dementia 7: 338-355 (2011). It refers to an intermediate stage between the expected cognitive decline as a result of aging and the more pronounced decline due to dementia. The symptoms of MCI typically include problems with memory, language, thinking and judgment, but the problems generally are not significant enough to interfere with daily life and activities, and the patients are often aware that their cognitive function is slipping. MCI is believed to increase the risk of developing dementia, including Alzheimer's disease, especially when the predominant symptom of MCI is memory impairment (see, e.g., Grundman et al., Arch. Neurol. 61 (1): 59-66 (2004)), and is likely caused in some instances by the underlying pathophysiology of Alzheimer's disease (see, e.g., Petersen, supra; Albert et al., Alzhermer's & Dementia 7: 270-279 (2011)).

“Neurodegeneration” means the progressive loss of structure or function of neurons. Neurodegenerative disorders, i.e., disorders which occur as a result of neurodegenerative processes, include but are not limited to Alzheimer's disease, Parkinson's disease, Huntington's disease, stroke, ALS, peripheral neuropathies, and other conditions characterized by damage, necrosis or loss of neurons, including for example central, peripheral, or motor neurons.

Clinical criteria for categorizing the stage of a specific neurodegenerative disorder have been established and documented. For example, a subject suffering form an early stage or mild Alzheimer's disease typically experiences memory loss for recent events, difficulty with problem solving, complex tasks and sound judgments, changes in personality, difficulty organizing and expressing thoughts, and getting lost or misplacing belongings (see the online information from Mayo Clinic). As the disease progresses, the patient generally grow more confused and forgetful and begin to need help with daily activities and self-care. Symptoms of Parkinson's disease include tremor, slowed motion (bradykinesia), rigid muscles, impaired posture and balance, loss of automatic movements (such as blinking, smiling and swinging your arms when you walk), speech changes (see the online information from Mayo Clinic). If the signs and symptoms are only on one side of the body, symptoms are mild and not disabling, usually have tremors in one limb, and changes in posture, locomotion, and facial expression are noticed by friends, it is considered to be an early stage Parkinson's disease (online information from eMedTV, by Arthur Schoenstadt, MD). Huntington's disease (HD) is genetically dominant disorder that affects muscle coordination and leads to cognitive decline and dementia. The earliest symptoms generally include a lack of coordination, and an impaired gait and balance. As the disease progresses, uncoordinated, jerky body movements become more apparent, along with a decline in mental abilities and behavioral and psychiatric problems.

Other conditions for which the disclosed methods are effective to treat include damage to the nervous system due to trauma, burns, and dysfunction or injury of certain organs such as lung, kidney or pancreas, as well as peripheral neuropathies associated with certain conditions, such as neuropathies associated with diabetes, for example, so long as the condition being treated is mild, which may be characterized by loss of structure or function of neurons, e.g., loss of synaptic spines and/or neuronal processes, without extensive apoptosis of neurons.

By “treating” is meant, at the molecular level, effective inhibition of the interactions between proNT and p75^{NTR}/SorCS2 expressed on neurons, to reduce, slow down the progression of, and/or prevent further development of neuronal growth cone collapse, reduction of synaptic spines, and/or or neurite pruning. Treatment should result in ameliorating the symptoms of the disorder, slowing down the progression of the disorder, and/or prevention of progression of the disorder.

Subjects which can be treated in accordance with the present methods include any mammalian subject, particularly a human subject.

Proneurotrophins (“ProNT”)

Proneurotrophins (“proNT”) are members of a well defined family. For example, the molecular masses of monomeric, unglycosylated proneurotrophins, including the N-terminal signal sequence, range from approximately 22 to approximately 30 kDa. The isoelectric points of the proneurotrophins range from approximately 8 to approximately 9. Most characteristically, the proneurotrophins are cleaved by proteases at or near the consensus cleavage site of the furin type to produce a mature neurotrophin.

Members of the proneurotrophins include proNGF, proBDNF, proNT-3, and proNT-4/5. The molecular masses of monomeric, unglycosylated proNGF, proBDNF, proNT-3, and proNT-4/5 are approximately 27.0, 27.8, 29.4, and 22.4 kDa, respectively. The GenBank accession numbers of human proNGF, proBDNF, proNT-3, and proNT-4/5 are AAA5993 1 (SEQ ID NO: 1), AAA69805 (SEQ ID NO: 2), AAA59953 (SEQ ID NO: 3), and AAA60154/AAA20549 (SEQ ID NO: 4), respectively. These sequences are also fully described in U.S. Pat. No. 7,507,799, which is incorporated herein by reference.

Antagonists of the proNT-p75^{NTR}/SorCS2 Ligand-Receptor System

In accordance with the present invention, administration of an antagonist of the proNT-p75^{NTR}/SorCS2 ligand-receptor system inhibits interactions between proNT and p75^{NTR}/SorCS2 expressed on neuronal cells, thereby controlling, reducing and/or preventing unwanted neuronal growth cone collapse and/or neurite pruning, and hence permitting treatment of early stage or mild neurological disorders.

The term “antagonist” as used herein, refers to a molecule that inhibits the expression level of a component of the proNT-p75^{NTR}/SorCS2 ligand-receptor system on neurons (“expression antagonist”); or alternatively, inhibits the interaction or binding between the components of the proNT-p75^{NTR}/SorCS2 ligand-receptor system expressed on neurons (“binding antagonist”), thereby reducing the amount, formation, function, and/or downstream signaling of this ligand-receptor system.

A molecule is considered to inhibit the expression level of a component of the proNT-p75^{NTR}/SorCS2 system if the molecule causes a significant reduction in the expression (either at the level of transcription or translation) of the component. Similarly, a molecule is considered to inhibit the binding between the components of the proNT-p75^{NTR}/SorCS2 ligand-receptor system if the molecule causes a significant reduction in the binding between the components and the ligand-receptor complex formed, which causes a significant reduction in downstream signaling and functions mediated by the ligand-receptor system, e.g., inactivation of the actin-bundling protein fascin and the dissociation of the Rac activator Trio from p75^{NTR}/SorCS2 and concomitant inactivation of Rac. A reduction is considered significant, for example, if the reduction is at least about 25%, and in some embodiments at least about 50%, and in other embodiments at least about 75%, 85%, or 95%.

A binding antagonist can act in two ways. A binding antagonist can compete with a proNT ligand for the receptors thereby interfering with, blocking or otherwise preventing the binding of the proNT ligand to p75^{NTR} and/or SorCS2. This type of antagonist, which binds the receptor but does not trigger the expected signal transduction, is also known as a “competitive antagonist” and can include, for example, an oligopeptide designed based on a proNT sequence, or an antibody directed to SorCS2 or p75^{NTR}. Alternatively, a binding antagonist can bind to and sequester a proNT ligand, with sufficient affinity and specificity to substantially interfere with, block or otherwise prevent binding of proNT to p75^{NTR} and/or SorCS2. This type of antagonist is also known as a “neutralizing antagonist”, and can include, for example, an antibody or aptamer directed to a proNT which binds specifically to a proNT.

An antagonist can also be characterized based on the target molecule which the antagonist is intended to antagonize. For example, a proNT antagonist refers to a molecule which inhibits or reduces the expression of a proNT or interferes with, blocks or otherwise prevents the interaction or binding of a proNT to p75^{NTR} and/or SorCS2. On the other hand, a SorCS2 antagonist refers to a molecule which inhibits or reduces the expression of SorCS2; or interferes with, blocks or otherwise prevents the interaction between SorCS2 and one or more proNT and/or p75^{NTR}; and a p75^{NTR} antagonist refers to a molecule which inhibits or reduces the expression of p75^{NTR}; or interferes with, blocks or otherwise prevents the interaction between p75^{NTR} and one or more proNT and/or SorCS2.

ProNT Antagonists

In one embodiment, a proNT antagonist is administered to achieve inhibition of interactions between a proNT and p75^{NTR}/SorCS2 expressed on neuronal cells, thereby controlling, reducing and/or preventing unwanted neuronal growth cone collapse and/or neurite pruning.

As disclosed herein, a proNT antagonist can be a neutralizing antibody that is specific for a particular proNT, an aptamer, an oligopeptide, a small molecule compound, or a nucleic acid molecule which reduces the level or activity of a proNT mRNA, for example.

In a specific embodiment, a proNT antagonist is a neutralizing antibody that is specific for a proNT, such as any one of proNGF, proBDNF, proNT-3, or proNT-4/5.

In this disclosure, a molecule (such as an antibody or aptamer) that is specific for a proNT is a molecule that binds with substantially greater affinity, and in some embodiments, binds nearly exclusively to the relevant proNT, relative to the mature version of the proNT and other proNT molecules. By “substantially greater affinity” it is meant that the binding affinity (Kd) of a molecule for a proNGF is at least 5 fold, 10 fold, 50 fold, 100 fold, or 1000 fold or greater, of the binding affinity of the molecule for the mature neurotrophin or other proneurotrophins.

In one particular embodiment, an antibody specific for a proNT is an antibody directed to the prodomain of the proNT. The prodomains of human proNGF, proBDNF, proNT-3, and proNT-4/5 are described in U.S. Pat. No. 7,507,799, and correspond to amino acid residues 1-approximately 117 of human proNGF, 1 to approximately 124 of human proBDNF, and 1 to approximately 134 of human proNT-3, and 1 to approximately 76 of human proNT-4/5, respectively. The prodomains of proNTs are distinct from each other, making it unlikely that antibodies raised against the prodomain of a proNT will cross-react with other proneurotrophins, or with mature neurotrophin. If desirable, the specificity of an anti-proNT antibody can be confirmed by using assays known in the art. For a given proNT, the pro-domain is highly conserved across species. For example, significant regions of identity are present within the pro-domain of proNGF from human, macque monkey, pig, dog, rat, and mouse, enabling the generation of a spectrum of antibodies to the prodomain directed to different regions, motifs, tertiary structures, or epitopes of the prodomain of proNGF.

The term “antibody” as used herein includes intact immunoglobulin molecules, as well as molecules that include an antibody hypervariable region that binds specifically to an intended antigen, with or without an antibody constant region. The hypervariable region can include an entire antibody variable region. Thus, an antibody molecule that includes an antibody hypervariable region can be an intact antibody molecule, antibody fragments (including single chain antibodies) which retain the antigen binding specificity of intact antibodies, as well as chimeric and humanized antibodies. The antibody can be polyclonal or monoclonal, and can be of any class of immunoglobins, such as: IgG, IgM, IgA, IgD or IgE, and the subclass thereof.

Suitable antibodies can be produced in a non-human mammal, including for example, rabbits, rats, mice, horses, goats, camels, or primates. Monoclonal antibodies produced from a non-human mammal can be humanized to reduce the immunogenicity for use in humans following techniques documented in the art. For example, to humanize a monoclonal antibody raised in mice, one approach is to make mouse-human chimeric antibodies having the original variable region of the murine mAb, joined to constant regions of a human immunoglobulin. Chimeric antibodies and methods for their production are well known in the art. See, e.g., Cabilly et al., European Patent Application 125023 (published Nov. 14, 1984); Taniguchi et al., European patent Application 171496 (published Feb. 19, 1985); Morrison et al., European Patent Application 173494 (published Mar. 5, 1986); Neuberger et al., PCT Application WO 86/01533, (published Mar. 13, 1986); all of which are incorporated herein by reference. Alternatively, humanized antibodies can be made to by including constant regions of a human immunoglobulin, and additionally, substituting framework residues of the variable regions of a non-human antibody with the corresponding human framework residues, either leaving the non-human CDRs substantially intact, or even replacing the CDR with sequences derived from a human genome. See, e.g., Maeda et al., Hum. Antibod. Hybridomas 2: 124-134, 1991, and Padlan, Mol. Immunol. 28: 489-498, 1991. As an additional alternative, human antibodies can be produced from transgenic animals (e.g., transgenic mice) whose immune systems have been altered to correspond to human immune systems. An example of such a mouse is the so-called XenoMouse™ (Abgenix, Freemont, Calif.), described by Green, “Antibody Engineering via Genetic Engineering of the Mouse: XenoMouse Stains are a Vehicle for the Facile Generation of Therapeutic Human Monoclonal Antibodies,” J. Immunol. Methods 10; 231(1-2):11-23 (1999).

In another specific embodiment, a proNT antagonist is an aptamer that binds specifically to a particular proNT.

Aptamers are molecules, either nucleic acid or peptide, that bind to a specific target molecule. Nucleic acid aptamers are generally short strands of DNA or RNA that have been engineered through repeated rounds of in vitro selection known as SELEX (systematic evolution of ligands by exponential enrichment) to bind to various molecular targets. Peptide aptamers can be selected using various systems, most frequently through the yeast two hybrid system. Peptide aptamers generally consist of a variable peptide loop (typically composed of ten to twenty amino acids), attached at both ends to a protein scaffold. This double structural constraint greatly increases the binding affinity of the peptide aptamer to levels comparable to an antibody.

In still another specific embodiment, a proNT antagonist is an oligopeptide or a small molecule compound that binds to the receptors of the proNT (i.e., the p75 receptor and/or SorCS2) thereby blocking the binding of proneurotrophin to its receptors, but does not lead to the downstream signaling or biological activity triggered by binding of proneurotrophin to p75^{NTR}/SorCS2.

Such small molecules and oligopeptides can be discovered by methods well known in the art. Typically, discovering such molecules involves providing a cell that expresses p75^{NTR} and/or SorCS2, providing a small molecule or oligopeptide to be tested, and determining whether the small molecule or oligopeptide to be tested binds to p75^{NTR} and/or SorCS2 and, optionally, results in the biological activity caused by binding of a proNT to p75^{NTR}/SorCS2. If the molecule binds with high affinity to p75^{NTR} and/or SorCS2, it is a candidate for use in the present method to limit neurite pruning or neuronal loss. If the molecule binds to p75^{NTR} and/or SorCS2 with high affinity and blocks binding of a particular proNT or even several proNTs to p75^{NTR}/SorCS2, it is a stronger candidate. If, in addition to blocking binding, the molecule also fails to cause the biological activity expected from activating p75^{NTR}/SorCS2, the molecule is a candidate for pre-clinical or clinical trials.

The oligopeptide has at least approximately four amino acid residues, and in some embodiments at least approximately five amino acid residues, and in other embodiments at least approximately six amino acid residues. The maximum number of amino acid residues is not important, as long as the oligopeptide has the desirable properties mentioned above. The oligopeptide may be linear or cyclic.

Some examples of oligopeptides include:

(SEQ ID NO: 5)
S/T-P/S-R-V-(Z)z
(SEQ ID NO: 6)
S/T-P/S-R-V-L/M/V-(Z)z
(SEQ ID NO: 7)
S/T-P/S-R-V-L/M/V-F/L-(Z)z
(SEQ ID NO: 8)
S/T-P/S-R-V-L/M/V-F/L-S-(Z)z

wherein Z represents any alpha amino acid and z represents any number from 0 to approximately 20, preferably from 0 to approximately 10, and more preferably from 0 to approximately 5. Any of these oligopeptides may be cyclic.

Small molecules include organic compounds, organometallic compounds, salts of organic and organometallic compounds, saccharides, amino acids, and nucleotides. Small molecules typically have molecular weights less than approximately 1000 Daltons, in some embodiments less than 800 Daltons. Small molecules include compounds that are found in nature as well as synthetic compounds.

In another specific embodiment, a proNT antagonist administered is a nucleic acid molecule which reduces the level or activity of a proNT mRNA. Such nucleic acid molecule includes an antisense RNA, a siRNA, a miRNA (or “microRNA”) or a transgene which codes for and is capable of expressing any such RNA molecule in the target tissue of a recipient. An antisense RNA is an RNA molecule that is complementary to endogenous mRNA and blocks translation from the endogenous mRNA by forming a duplex with the endogenous mRNA. siRNAs are small (typically 20-25 nucleotides in length) double-stranded RNAs which are known to be involved in the RNA interference pathway and interfere with the expression of a specific gene. Given the sequence of a target gene, siRNAs can be designed, and made either synthetically or in cells from an exogenously introduced vector (e.g., a plasmid) to achieve suppression of expression of a gene of interest. Similar to siRNAs, miRNAs are also small RNA molecules (generally about 21-22 nucleotides) that regulate gene expression. miRNAs are processed from long precursors transcribed from non-protein-encoding genes, and interrupt translation through imprecise base-pairing with target mRNAs. miRNA can be designed and introduced to cells or tissues to target and suppress the expression of a gene of interest (proNT, SorCS2 or p75^{NTR}) using techniques documented in the art. Modulation of miRNA can be accomplished by viral-mediated delivery of pro-miRNA or decoymiR or by delivery in plasma (as examples, Cordes K R, et al, Nature 460:705 (2009); Caporali A, et al., Circulation 123:282, (2011); Castoldi, M, J., Clin Invest. 121:1386 (2011); Vickers, K C et al., Nat Cell Biol 13: 423 (2011)).

SorCS2 Antagonist

In a further embodiment, a SorCS2 antagonist is administered to achieve inhibition of interactions between proNT and p75^{NTR}/SorCS2 expressed on neuronal cells, thereby controlling, reducing and/or preventing unwanted neuronal growth cone collapse and/or neurite pruning.

As disclosed herein, a SorCS2 antagonist can be an antibody, an aptamer, an oligopeptide, a small molecule compound, or a nucleic acid molecule which reduces the level or activity of the SorCS2 mRNA.

In a specific embodiment, a SorCS2 antagonist is an antibody that binds specifically to SorCS2 and inhibits the interaction of SorCS2 with one or more proNT and/or p75^{NTR}. An antibody that is specific for SorCS2 is an antibody that binds with substantially greater affinity, and in some embodiments, binds nearly exclusively to SorCS2, relative to other members of the sortlin family such as sortlin. By “substantially greater affinity” it is meant that the binding affinity of an antibody for SorCS2 is at least 5 fold, 10 fold, 50 fold, 100 fold, or 1000 fold or greater, of the binding affinity of the antibody for other members of the sortlin family.

In one particular embodiment, a SorCS2-specific antibody is directed to the ectodomain of SorCS2 (amino acids 20-1078 of human SorCS2). In certain embodiments, a SorCS2-specific antibody is specifically directed to specific motifs or epitopes within the ectodomain, such as the cystein-rich domain (amino acid residues 611-750 of human SorCS2), or the 10 bladed propeller domains (amino acids 45-610). The amino acid sequence of human SorCS2 is set forth in SEQ ID NO: 9 (Accession No. NP_{—}065828).

Similar to proNT antagonists as described above, SorCS2 antagonists are not limited to antibodies, but also include nucleic acid or peptide aptamers that bind specifically to SorCS2 and inhibit its interaction with proNT and/or p75^{NTR} oligopeptides or small molecule compounds that block the interaction of SorCS2 with proNT and/or p75^{NTR} as well as nucleic acid molecules (such as antisense, siRNA, or miRNAs) which reduce the level or activity of the SorCS2 mRNA.

p75^{NTR} Antagonist

In a further embodiment, a p75^{NTR} antagonist is administered to achieve inhibition of interactions between proNT and p75^{NTR}/SorCS2 expressed on neuronal cells, thereby controlling, reducing and/or preventing unwanted neuronal growth cone collapse and/or neurite pruning.

As disclosed herein, a p75^{NTR} antagonist can be an antibody, an aptamer, an oligopeptide, a small molecule compound, or a nucleic acid molecule which reduces the level or activity of the p75^{NTR} mRNA.

In a specific embodiment, a p75^{NTR} antagonist is an antibody that binds specifically to p75^{NTR} and inhibits the interaction of p75^{NTR} with one or more proNT and/or SorCS2. p75^{NTR} is well characterized in the art and is known to be a member of the tumor necrosis factor receptor (TNRF) family. The sequences of mammalian (including human) p75^{NTR} molecules are available to those skilled in the art for generating an antibody suitable for use in this invention.

In another embodiment, a p75^{NTR} antagonist can be any one of a nucleic acid or peptide aptamer that binds specifically to a p75^{NTR} and inhibits its interaction with proNT and/or SorCS2; an oligopeptide or small molecule compound that blocks the interaction of a p75^{NTR} with proNT and/or SorCS2; or a nucleic acid molecule (such as an antisense, siRNA, or miRNA molecule) which reduces the level or activity of the a p75^{NTR} mRNA.

Cocktails of Antagonists

Also provided herein is a cocktail of more than one antagonist molecule, which is also suitable for administration. The cocktail may, for example, include one or more antibody molecules, one or more aptamer molecules, one or more oligopeptides or small molecules, or various combinations thereof. The cocktail can also include any combination of one or more proNT antagonists, one or more SorCS2 antagonists, and one or more p75^{NTR} antagonists.

Administration

An antagonist or a cocktail of antagonists is administered to a subject in need of the treatment in order to control, reduce or prevent further development of unwanted neuronal growth cone collapse and/or neurite pruning. Suitable subjects include, for example, subjects suffering early stage or mild neurological disorders including mild cognitive impairment, or early stage or mild neurodegenerative disorders.

An antagonist can be combined with a pharmaceutically acceptable carrier in any convenient and practical manner, e.g., by admixture, solution, suspension, emulsification, encapsulation, absorption and the like, and can be made in formulations such as tablets, capsules, powder, syrup, suspensions that are suitable for injections, implantations, inhalations, ingestions or the like.

As used herein, a pharmaceutically acceptable carrier includes any and all solvents, dispersion media, isotonic agents and the like. Except insofar as any conventional media, agent, diluent or carrier is detrimental to the recipient or to the effectiveness of the active ingredients contained therein, its use in practicing the methods disclosed herein is appropriate. The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives and the like, or combinations thereof.

The concentration of an antagonist in formulations may range from as low as about 0.1% to as much as 15 or 20% by weight and can be selected based on the nature of the particular antagonist used, the mode of administration selected, among other considerations. Thus, a typical formulation for injection could be made up to contain 1 mL sterile buffered water of phosphate buffered saline and 1-1000 mg, possibly 10-100 mg, of an antagonist such as an antibody-based antagonist, for example.

Depending on the nature of the antagonist or the conditions of the subject, a pharmaceutical formulation containing an antagonist, can be given to the subject by standard routes, including ingestion, injections via an intravenous, intraperitoneal, subcutaneous, transdermal, or intramuscular route, delivery through an intranasal or sublingual route, delivery to the cerebral spinal fluid via a needle or catheter, for example. Where appropriate, delivery of an antagonist can be facilitated by concomitant use of agents or systems that enhance delivery across the blood brain barrier, for example, the use of mannitol, vasoactive substances such as bradykinin, endogenous transport systems including carrier-mediated transporters such as glucose and amino acid carriers, and nanoparticles.

The amount of antagonist administered to be effective may depend on the condition of the patient (e.g., age, body weight and health) and state of the disease. The precise amount of an antagonist to be effective can be determined by a skilled physician.

The present description is further illustrated by the following examples, which should not be construed as limiting in any way. The contents of all cited references (including literature references, issued patents, and published patent applications as cited throughout this application) are hereby expressly incorporated by reference.

### EXAMPLES

### Example 1

### Experimental Procedures

Materials.

The LifeAct sequence (RIEDL, J. et al., Nat Methods 5:605-607 (2008)) was cloned into the pEGFP-N1 backbone (Clontech) using the BglII and BamHI restriction sites. Trio constructs were described previously (FERRARO, F. et al., Mol Biol Cell 18:4813-4825 (2007)), human sortilin cDNA or human SorCS2 cDNA was subcloned into the pcDNA3.1 hygro expression vector (Invitrogen). A Myc-tag was inserted three residues after the furin site using PCR. FascincDNA was kindly provided by Prof. Xin-Yun Huang (CHEN, L. et al., Nature 464:1062-1066 (2010)) and was subcloned into pcDNA 3.1 using the BamHI and EcoRI sites. Following primary antibodies were used: anti-actin and anti-HA (Sigma), anti-fascin and anti-Rac1 (Millipore), anti-p75^{NTR} (9651 (HUBER, L. J. et al., Dev Biol 167:227-238 (1995)); and R&D Systems), anti-sortilin and anti-SorCS2 (R&D Systems), anti-Trio (Santa Cruz C20; and CT-35 (MCPHERSON, C. E. et al., Gene 284:41-51 (2002)), anti-Myc and control IgGs (Santa Cruz). Fluorescent secondary antibodies were from Invitrogen, HRP-coupled secondary antibodies from Sigma and Amersham. Anti-HA agarose was from Roche Diagnostics, anti-Myc (9E10) agarose and control IgG agarose was from Santa Cruz. All chemicals were from Sigma unless indicated otherwise. ProNGF was produced in Sf9 cells and was purified as described (FENG, D. et al., Journal of Mol Biol 396:967-984 (2010)), and proBDNF was collected from supernatants of 293 cells transfected with pcDNA encoding murine proBDNF as described (TENG, H. K. et al., Journal of Neuroscience 25:5455-5463 (2005)).

Cell Treatments.

To assay which pathways are involved in filopodial collapse, 5-10 ng/ml proNGF, 10 ng/ml proBDNF, 25-50 ng/ml BDNF (Peprotech) or 25-100 ng/ml NGF (Harlan) was added to E15 primary hippocampal neurons for 20 min before fixation. In some cases, cells were pretreated with 100 nM K-252a (Calbiochem) for 20 min, 100 nM Gö6976 (Calbiochem) for 1 hour, 5 μM EHT1864 (Sigma) for 30 min or 100 nM ITX3 (ChemBridge Corporation, cat. no. 6007262) for 45 min. To block SorCS2, cells were incubated with 20 μg/ml anti-SorCS2 or control IgG for 20 min on ice before proNGF addition at 37° C.

Immunofluorescence.

DIV3 hippocampal neurons were fixed in 4% paraformaldehyde/20% sucrose for 10 min. The fixative was quenched with 50 mM NH₄Cl in PBS for 5 min and cells were permeabilized with 0.1% TritonX-100 in PBS for 2 min. Alternatively, cells were fixed with ice-cold methanol for 5 min. Coverslips were blocked with 10% normal donkey serum, 2% bovine serum albumin and 0.25% fish skin gelatin in Tris-buffered saline for 30 min, incubated with primary antibodies diluted in blocking solution for 30 min, washed three times with Tris-buffered saline/0.25% fish skin gelatin, incubated with secondary antibodies mixed with Hoechst in blocking buffer for additional 30 min, washed and mounted using Mowio1488. Cells were imaged using a LSM510 laser-scanning confocal microscope equipped with a 40× Plan Neofluor NA1.3 DIC oil-immersion objective (Carl Zeiss Microimaging). Images were processed using LSM510 software (Zeiss) and ImageJ (NIH).

Live Imaging and Data Analysis.

Cells were plated on gridded glass bottom dishes (MatTek Corporation) and imaged at DIV3 in phenol red-free Neurobasal medium (Invitrogen) supplemented with 30 mM HEPES-NaOH, pH7.4 using an Olympus IX71 inverted microscope driven by IPLab software (BD Biosciences) and equipped with a 60× PlanApoN objective (NA 1.42), a Hamamatsu EM-CCD camera and a heated stage maintained at 37° C., or driven by softWoRX software (version 3.7.1, DeltaVision), and equipped with a CoolSnap HQ2 camera (Photometrics) and an environmental chamber maintained at 37° C. Images were taken every 15 seconds. After live imaging cells were fixed, counterstained for p75, located by grid number and examined for p75 expression. All movies were exported and processed using IPLab (BD Biosciences), softWoRX (version 3.7.1, DeltaVision) and ImageJ (NIH) software. Filopodia length and speed were both analyzed using ImageJ (NIH) software. For this, two times ten consecutive frames, spaced 5-10 min apart, were analyzed before and again after treatment. In the length analysis, a line was drawn along an individual filopodium and measured at every time point, and the measurements were averaged before and after treatment. For the speed analysis, the filopodial tip was tracked using the ImageJ Manual Tracking plugin. Up to four individual filopodia were analyzed per movie, and a minimum of four movies taken from separate experiments were analyzed per condition.

Co-Immunoprecipitations.

HT-1080 cells were transfected with HA-p75^{NTR} and myc-SorCS2. 24-48 hours later, cells were treated with 25 ng/ml proNGF for 20 min where indicated. Cells were lysed with lysis buffer (50 mM Tris-HCl pH 8.0, 140 mM NaCl, 2 mM EDTA, 1% NP40, 10% glycerol) supplemented with protease inhibitors and complexes were immunoprecipitated using anti-HA agarose. To map the Trio domain required for the interaction with p75^{NTR}, 293T cells were transfected with HA-p75^{NTR} and constructs of the individual Trio domains. After 24 hours, cells were lysed as described above, and complexes were immunoprecipitated using anti-HA agarose or anti-Myc agarose as indicated.

Rac Activity Assay.

Rac activity assays were performed as described previously (NEUBRAND, V. E. et al., Journal of Cell Science 123:2111-2123 (2010)). DIV2 cortical neurons were stimulated with 25 ng/ml proNGF for 20 min. Cells were lysed in lysis buffer supplemented with 10 mM MgCl, lysates were cleared by centrifugation at 9000 g for 1 min, and cleared lysates were incubated with GST or GST-Pak-CRIB beads for 30 min at 4° C. Beads were washed and isolated active Rac was analyzed by Western blot. As a control, extra lysates were incubated with 0.1 mM GDP or 0.1 mM GTPγS for 30 min at room temperature before incubation with GST-Pak-CRIB beads. Western blots were analyzed by densitometry using ImageJ software, and isolated active Rac was normalized to the input.

Perfusion & Preparation of Sections for Immunofluorescence Staining.

Postnatal day 35 (P35) mice were anesthetized with pentobarbital, transcardially perfused with 0.9% saline followed by 3% paraformaldehyde. After infiltration in sucrose, brains were embedded in sucrose/O.C.T and sectioned at 10 μm. For the staining, sections were incubated in blocking buffer (5% BSA+0.1% Triton X-100) and in avidin/biotin blocking kit (Vector labs) at room temperature, then incubated with primary anti-HA antibody (1:500, Sigma) for 18 hr at 4° C. Slides were washed for 3×10 min with PBS and incubated with secondary biotinylated goat anti-rabbit antibody (1:400, Jackson ImmunoResearch), and Cy3-conjugated streptavidin (1:800, Jackson Immunoresearch) was used to visualize the images on an Olympus BX51 microscope. For p75^{NTR} and SorCS2 staining, 6-week old frozen brain sections were blocked with 5% BSA+0.1% Triton-100 for 1 hr at RT, then incubated with either anti-p75^{NTR} antibody (1:1000, R&D system) or anti-SorCS2 antibody (1:350, R&D system) O/N at 4° C. After washing with PBS, for p75^{NTR} staining, the sections were incubated with Alexa Flor 546 donkey anti-goat antibody (Invetrogen) for 1 hr at RT and mounted; for SorCS2 staining, sections were incubated with biotinylated donkey anti-sheep antibody (Jackson Immunoresearch) for 1 hr, and Cy3-conjugated streptavidin (Jackson ImmunoResearch) was used to visualize the images. Images were taken on a Zeiss LSM700 confocal microscope.

Measurement of Hippocampal Volume.

Hippocampal volumes were measured as described (BATH, K. G. et al., Magn Reson Imaging 27:672-680 (2009)). Briefly, animals were anesthetized with pentobarbital, and transcardially perfused with 0.9% saline, 0.1% sodium nitrite and 5% gadolinium-DTPA (Magnevist, Berlex Laboratories, Wayne, N.J., USA) followed by 4% paraformaldehyde solution and 5% Magnevist in PBS. The brains were then stored in 0.1M PBS containing 5% Magnevist for 3-7 days prior to imaging. A 3.0-T magnetic resonance imaging system (GE Medical Systems, Milwaukee, Wis., USA) equipped with 50 mT/m gradients operating at 150 mT/m per millisecond was used to image the brains. Images were analyzed by a blinded observer utilizing Osirix software (The Osirix Foundation, Geneva, Switzerland). For hippocampi, the external capsule, alveus of hippocampus and white matter were used as boundary landmarks.

Rapid Golgi Impregnation.

Golgi impregnation of all brains was conducted using the Golgi-Cox method. The solution was stored in a dark place at room temperature. Dissected whole brains were immersed in Golgi-Cox solution I in a glass bottle for 10 days at room temperature protected from lights. After the initial 12 hours of immersion, the solution was changed. After 10 days, brains were switched to 30% sucrose prepared in H₂O (15 ml/brain) for 4 days at 4° C. in a dark place (after the initial 12 hours, the solutions were changed). Brains were then cut on a vibratome (150 μm sections). For coronal sections, entire brains were cut into two parts at the groove between cerebral cortex and cerebellum, and directly glued on the sample plate. Serial sections were immediately mounted onto 0.3% gelatin coated slides. Prior to complete drying, sections were brushed with sucrose, and allowed to air dry for at least 2 days. To visualize the stained neurons, slides were then immersed in H2O for 3×10 min with gentle shaking, then transferred into Golgi-Cox solution II for 5-10 min at room temperature followed 3×5 min raise in H₂O. Slides then went through a dehydration process and were cleared with Histoclear (3×5 min), and DPX mounting medium was used to coverslip the sections.

Golgi Tracing.

Golgi impregnated brain sections were numbered blindly prior to quantitative analysis. Hippocampal dentate gyrus (DG) neurons were traced in the dorsal hippocampus. The selected DG neurons for dendritic arborization analysis must satisfy the following criteria: 1) single cell body having primary dendrites growing out from the soma, and relatively isolated from the neighboring neurons; 2) having intact dendrites with consistent impregnation along the dendrites. 20-30 neurons from each animal were traced under 40× magnification using Neurolucida software. The morphological traits of cells (Sholl analysis and Fractal dimension analysis) were analyzed using Neuroexplorer. Prism 4.0 was used to process the data and for statistical analyses (two-way ANOVA).

Cell Culture and Transfection.

Primary hippocampal and cortical neurons were isolated from E15 C57BL/6 mouse embryos as previously described (PEREIRA, D. B. et al., Journal of Neuroscience 27:4859-4869 (2007)). Briefly, neurons were dissociated by incubation with 0.05% trypsin at 37° C. for 8 min followed by trituration with fire-polished glass Pasteur pipettes. Cells were plated on poly-D-lysine coated dishes and grown in Neurobasal medium containing B27, 0.5 mM glutamine (all Invitrogen) and 10 μM 5-fluorodeoxyuridine. Hippocampal neurons were transfected at DIV2 using Lipofectamine2000 (Invitrogen). For coverslips in a 24-well plate, 0.5 μg plasmid were mixed with 0.5 μl Lipofectamine2000, and for glass bottom dishes, 1.5 μg plasmid were mixed with 1.5 μl Lipofectamine2000. The complexes were added to the cells for 30-45 min, and then neurons were placed back into preconditioned media until analysis the following day. HT1080 cells were grown in DMEM/10% fetal bovine serum (both Invitrogen) and transfected using the AMAXA nucleofector (Lonza) according to manufacturer's instructions. 293FT cells were grown in DMEM/10% fetal bovine serum and transfected with Lipofectamine2000 according to manufacturer's instructions.

Purification of GST-Pak-CRIB.

GST and GST-Pak-CRIB proteins were expressed in E. coli BL21DE3 cells at 25° C. for 3 hours. Bacteria were disrupted by incubation with PBS supplemented with lysozyme on ice for 30 min and subsequent addition of 1 mM MgCl2, 0.1% TritonX-100 and 0.1 mg/ml DNAse for an additional 30 min. Bacterial lysates were cleared by centrifugation for 5 min at 3000 g and cleared lysates were incubated with glutathione sepharose. Beads were washed and the purity of the recombinant proteins was analyzed by Coomassie blue staining. 20 μg of sepharose coupled purified protein was used per reaction.

Generation of proBDNF-HA Knock-in Mice.

proBDNF-HA knock-in mice were generated by substituting one allele of the murine coding exon V of the bdnf gene with the murine exon V in which the furin cleavage site was mutated (RR-AA) and a HAepitope tag was added to the C-terminus. For this, the 129 genomic DNA (pLTM25, containing 16 kb BglII-BglII sequence) was digested using KpnI and Xba enzymes and subcloned into pBluescript (containing 3.5 kb short and 4.8 kb long arms). The hemagglutinin (HA) epitope tag was added by site-directed mutagenesis in-frame before the stop codon. The furin recognition site was mutated using site-directed mutagenesis (Stratagene) from RR to AA. In addition, flp and loxP sites surrounding a neomycin selectable cassette were engineered to mimic the gene-trap strategy utilized by the Baygenomics consortium. Briefly, a 1.7 Kb C57B1/6 PCR generated fragment containing the intron 1 and the first 7 amino acids of exon 2 of the mouse engrailed 2 gene (splice acceptor element) was placed upstream of a pGK/EM7/neobpA cassette. This cassette was introduced at ˜450 bp 5′ of the HA-encoding exon V, in a region of low homology between murine and human sequences. The targeting construct was electroporated into 129SvJ embryonic stem cells and diptheria toxin was used for negative selection. Positive clones were identified using Southern blotting. Chimera breeding enabled us to select three lines of probdnf-HA mice for further analysis. Mice carrying the probdnf-HA allele were crossed with the EIIa-Cre deleter strain (Jax Mice) to generate littermates expressing one probdnf-HA allele, and one endogenous bdnf allele (probdnf-HA/+). probdnf-HA mice were backcrossed more than 10 generations to C57B1/6. bdnfha mice were generated as described (YANG, J. et al., Nat Neuroscience 12:113-115 (2009)). bdnf+/+ and bdnf−/− mice were generated from intercrosses of bdnf+/− mice obtained from Jackson Laboratories.

Immunoprecipitation from Tissue Lysates.

Dissected tissues were minced and lysed in lysis buffer2 (1× Tris buffered saline (TBS), 1% NP-40, 1% TritonX-100, 1 mM PMSF, 10% glycerol, and protease inhibitor cocktail (Sigma)) for 30 min on ice. Lysates were further triturated using a 30 g needle, and supernatants were collected following centrifugation at 14,000 rpm for 5 min. Lysate samples were subjected to immunoprecipitation: first, samples were pre-cleared using protein A-Sepharose beads, and the supernatant was then incubated with anti-HA antibody for 18 h at 4° C. Blocked protein A-Sepharose beads (incubated with 5% BSA for 30 min, and then washed extensively with lysis buffer2) were added to the supernatant for 2 hr at 4° C., and immunoprecipitates collected by centrifugation for 5 min at 5000 rpm. Beads were washed in lysis buffer2, and immuno-complexes were resolved by SDS-PAGE. Following transfer, Western blots were developed using incubation with anti HA.11 antibody (Covance), then with HRP-conjugated secondary antibodies and developed with the ECL kit (Amersham).

Mass Spectrometry.

p75^{NTR} was immunoprecipitated from HT1080 cells stably expressing p75 or p75 and sortilin, immunoprecipitated proteins were resolved by SDS-PAGE and gels were stained with Coomassie Brilliant Blue (Bio-Rad). Bands that were only present in immunoprecipitates from cells expressing both p75^{NTR} and sortilin were excised from the gel along with a control band from the same location of the gel in the lane containing immunoprecipitate of p75^{NTR} without sortilin co-expression. Gel bands were cut into small pieces and destained in 25 mm ammonium bicarbonate in 50% acetonitrile, dehydrated with acetonitrile, and dried. The gel pieces were rehydrated with 10 ng/μl trypsin solution in 25 mM ammonium bicarbonate and incubated overnight at 37° C. Peptides were extracted twice with 5% formic acid in 50% acetonitrile followed by a final extraction with acetonitrile. Extracts were pooled, dried by vacuum centrifugation, and reconstituted in 5 μl of 0.1% formic acid, 2% acetonitrile for HPLC sample injection. Resuspended samples were loaded onto a Symmetry 5 μm particle, 180 μm×20 mm C18 precolumn (Waters), then washed 5 min with 1% acetonitrile in 0.1% formic acid at a flow rate of 20 μL/min. After washing, peptides were eluted and passed through an Atlantis 3 μm particle, 75 μm×100 mm C18 analytical column (Waters, Milford, Mass.) with a gradient of 1-80% Acetonitrile in 0.1% formic acid. The gradient was delivered over 120 min by a nanoACQUITY UPLC (Waters) at a flow rate of 250 mL/min, to a fused silica distal end-coated tip nano-electrospray needle (New Objective, Woburn, Mass.). Data were collected by a Q-TOF Premier mass spectrometer (Waters/Micromass) set for MS survey scans and automatic data-dependent MS/MS acquisitions. Raw LCMS/MS data were processed using ProteinLynx GlobalServer 2.2 software (Waters). A database containing the combined IPI mouse and rat sequences concatenated with their reverse sequences (186,384 total sequences) was searched using Mascot software (version 2.1, Matrix Science, London, United Kingdom) for protein identification. Search criteria included trypsin specificity with one missed cleavage allowed, methionine oxidation, and minimum precursor and fragment-ion mass accuracy of 50 ppm and 0.1 Daltons respectively.

### Example 2

### Generation of proBDNF-Expressing Mice

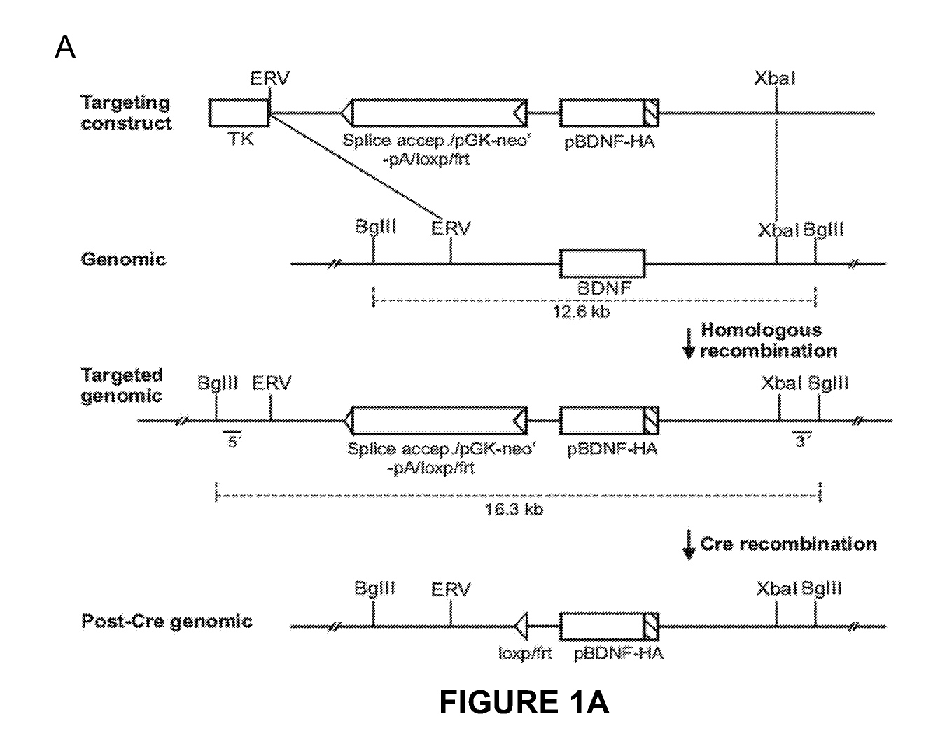

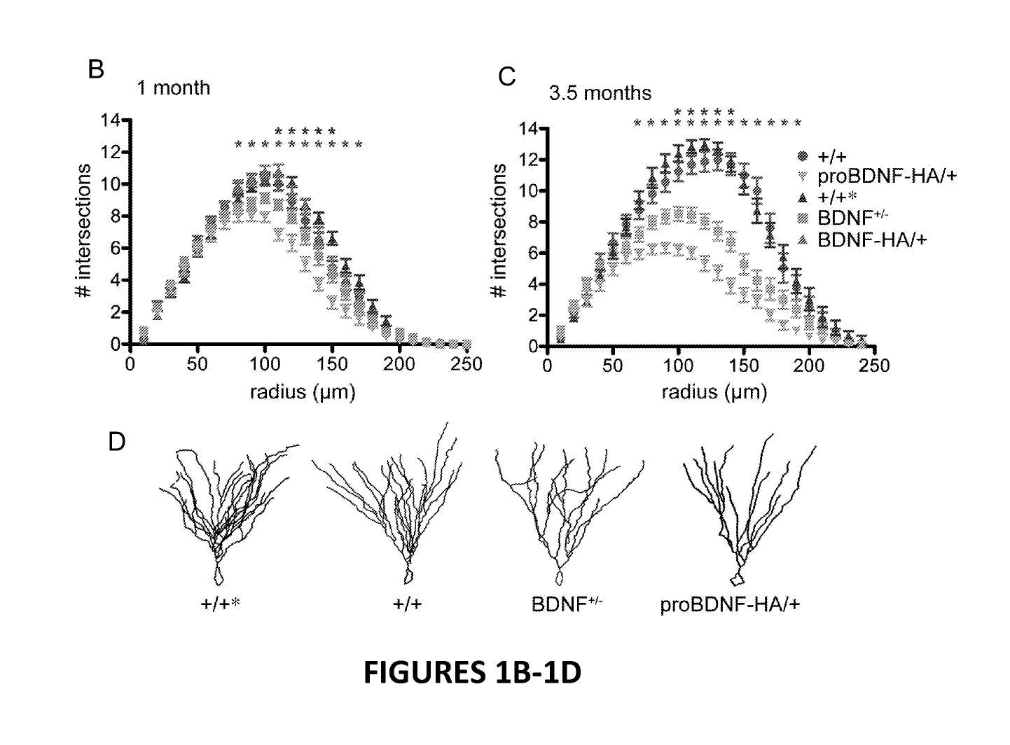

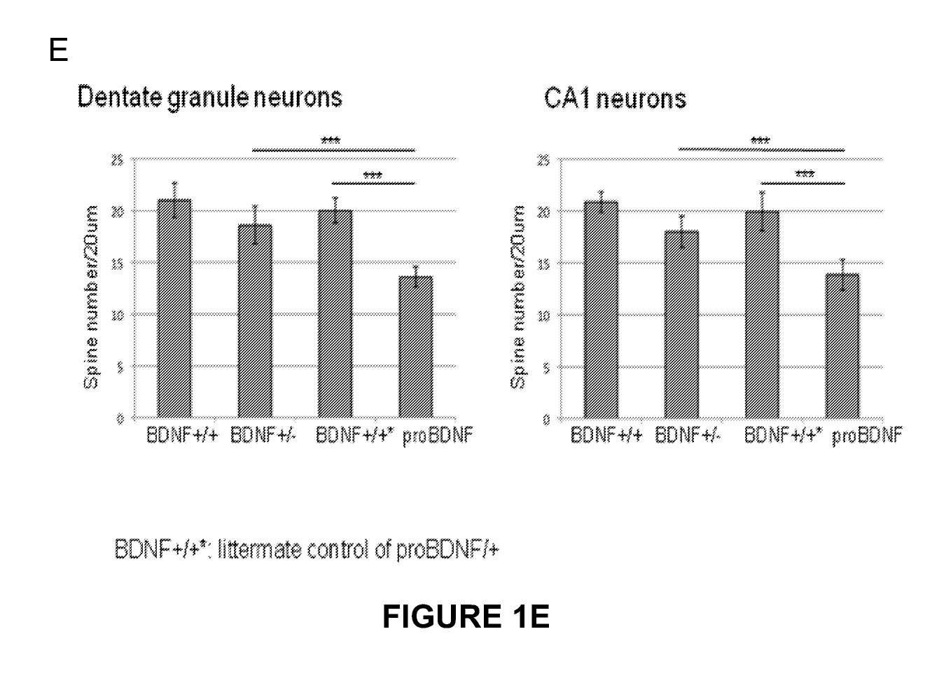

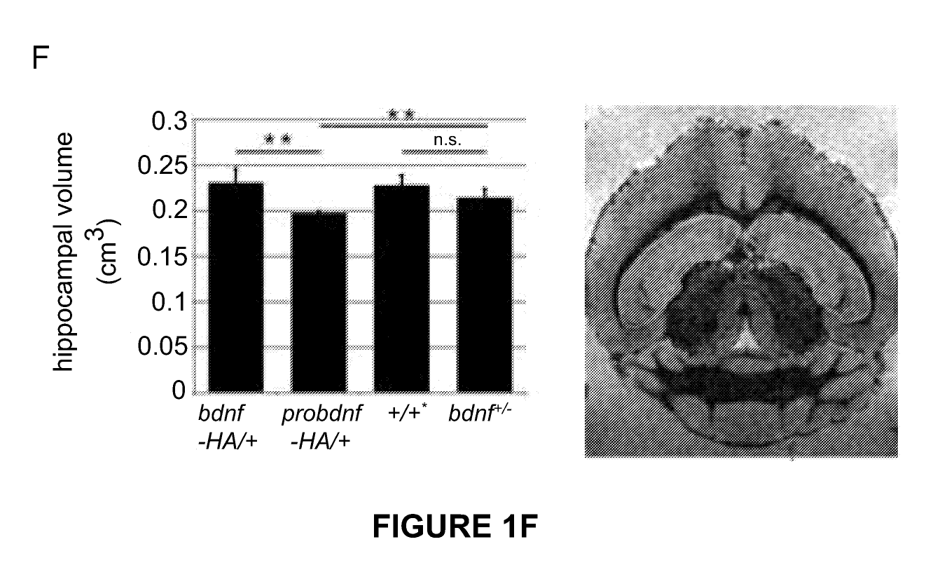

To evaluate the effects of proBDNF expression in vivo, the inventors generated a probdnf knock-in mouse, replacing one bdnf allele with probdnf with a mutated furin cleavage site, but leaving one endogenous allele intact to maintain viability (FIG. 1A). A C-terminal HA epitope tag was added to facilitate detection of the introduced allele (probdnf-HA). This approach was advantageous, since a substitution of both endogenous bdnf alleles with a cleavage-resistant probdnf would likely result in a lethal perinatal phenotype, as mature BDNF is required for vascular development (DONOVAN, M. J. et al., Development 127:4531-4540 (2000)). In addition, a probdnf transgene would fail to recapitulate the complex transcriptional and translational regulation of the bdnf locus (GREENBERG, M. E. et al., Journal of Neuroscience 29:12764-12767 (2009)). Because probdnf-HA/+ mice expressed only one endogenous bdnf allele, the inventors also analyzed animals haploinsufficient for bdnf (bdnf+/−) (LYONS, W. E. et al., Proc Natl Acad Sci USA 96:15239-15244 (1999)) in parallel, to assess gain-offunction phenotype(s) of probdnf. As an additional control, bdnf-HA knock-in mice in which the furin site was not mutated were also analyzed (YANG, J. et al., Nat Neuroscience 12:113-115 (2009)).

The inventors confirmed that total BDNF (proBDNF+mature BDNF) levels were comparable between probdnf-HA/+, bdnf-HA/+ mice and wild type littermates as measured by ELISA. To verify that mutation of the furin site resulted in elevated proBDNF levels, the inventors measured proBDNF after immunoprecipitation with HA-specific antibodies followed by Western blot analysis for HA reactivity. In the cortex (CT), hippocampus (HP) and cerebellum (CB) of probdnf-HA/+ mice, proBDNF-HA (˜33 kDa), but not processed mature BDNF-HA (˜14 kDa), was detectable, confirming that the introduction of the mutated sequence resulted in expression of intact proBDNF.

ProBDNF Expression Leads to Reduced Dendritic Arborization In Vivo.

To determine whether there were any anatomical deficits in the probdnf-HA/+ mice, the inventors analyzed the hippocampal region where BDNF is most highly expressed (Hofer et al., 1990). Immunofluorescence microscopy confirmed high levels of HA immunoreactivity in the CA3 and dentate gyrus regions of the hippocampi from probdnfHA/+ and bdnf-HA/+ mice (YANG, J. et al., Nat Neuroscience 12:113-115 (2009)). The inventors then performed Golgi staining of individual dentate gyrus neurons to assess dendritic complexity, comparing probdnf-HA/+ mice, haploinsufficient bdnf^{+/-} mice to account for the single allele of endogenous bdnf in the probdnf-HA/+ mice, bdnf-HA/+ mice to account for possible deficits due to the HA-tag, as well as the wild-type littermates of the bdnf^{+/-} mice (+/+*), and the wild-type littermates of the probdnf-HA mice (+/+). At one month of age, Sholl analysis demonstrated that probdnf-HA/+ mice displayed a significantly decreased complexity of dendritic arbors at a distance of 80 μm and further from the soma, when compared to their wild type littermates (+/+) (FIG. 1B). bdnf-HA/+ mice did not exhibit differences in dendritic complexity when compared to wild type animals, suggesting that the observed defect in probdnf-HA/+ was not due to the addition of the HA-tag (FIG. 1B). To assess whether the observed deficit in arborization in probdnf-HA/+ mice was a consequence of enhanced proBDNF or a lack of mature BDNF, we also analyzed dentate granule neurons of haploinsufficient (bdnf^{+/-}) mice. Bdnf^{+/-} mice displayed decreased dendritic complexity in the dentate gyrus compared to their wild type littermates (FIG. 1B). However, at distances of 110 μm and greater from the soma, probdnf-HA/+ mice showed a significant further decrease in complexity compared to bdnf^{+/-} mice, suggesting that local proBDNF expression yielded a gain-of function phenotype to contribute to deficits in dendritic arborization (FIG. 1B). These defects in dendritic complexity were even more pronounced in 3.5 months old mice (FIG. 1C; representative traces are shown in D), with probdnf-HA/+ mice displaying significantly reduced arborization at 70 μm and greater from the soma compared to their wild type littermates (FIG. 1C). Reduced arborization at distances of 100 μm and greater were observed in the probdnf-HA/+ mice compared to bdnf^{+/-} mice (FIG. 1C). The inventors also used fractal dimension analysis to quantify how completely a neuron fills its dendritic field. There was a significant decrease in dendritic complexity in dentate gyrus neurons from bdnf^{+/-} mice, and a further decrease in probdnf-HA/+ mice, as compared to wild type animals.

To analyze the spine density, Postnatal day 30 (P30) whole brains were dissected and subjected to Golgi staining as described above. Spine density of CA1 and DG neurons was measured on dendrites (per 20 μm) under 100× objective from 15 neurons of 3 animals per genotype. The total number of spines was presented per 20 micrometer of dendritic length. As shown in FIG. 1E, CA1 and DG neurons from proBDNFHA knock-in brains had less dendritic spine density compared to BDNF het and wild type neurons (T-test). All results are presented as means±SEM.

The above results indicate that augmented proBDNF expression impairs dendritic complexity early in postnatal development, and that these differences are maintained in later adult life. Therefore, proBDNF negatively influences dendritic development in vivo.

Since a pronounced defect in dendritic branching was observed, the inventors also measured the hippocampal volume of 11 month old probdnf-HA/+ mice using magnetic resonance imaging. See FIG. 1F. The inventors observed a 14.4% decrease in hippocampal volume in the probdnf-HA/+ mice when compared to the bdnf-HA/+ mice. These values were compared with the hippocampal volumes from age-matched bdnf^{+/-} mice. The bdnf^{+/-} mice displayed a trend but no significant decrease in hippocampal volume when compared to their wild type littermates, suggesting that proBDNF action contributed to the reduction of hippocampal volume.

Proneurotrophins Induce Growth Cone Collapse In Vitro.

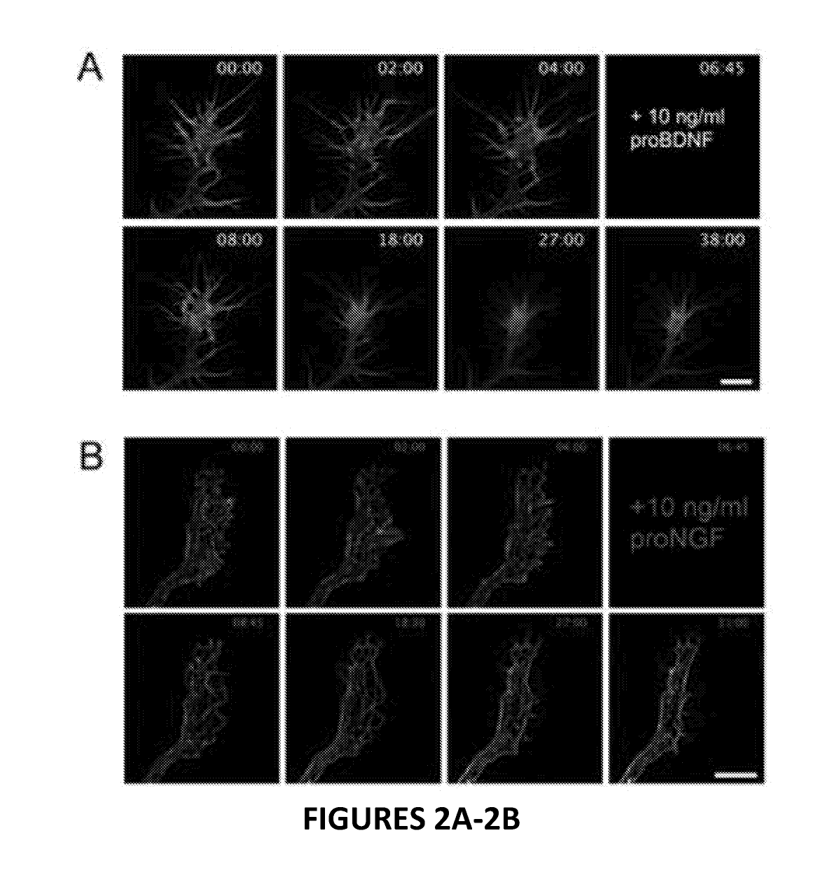

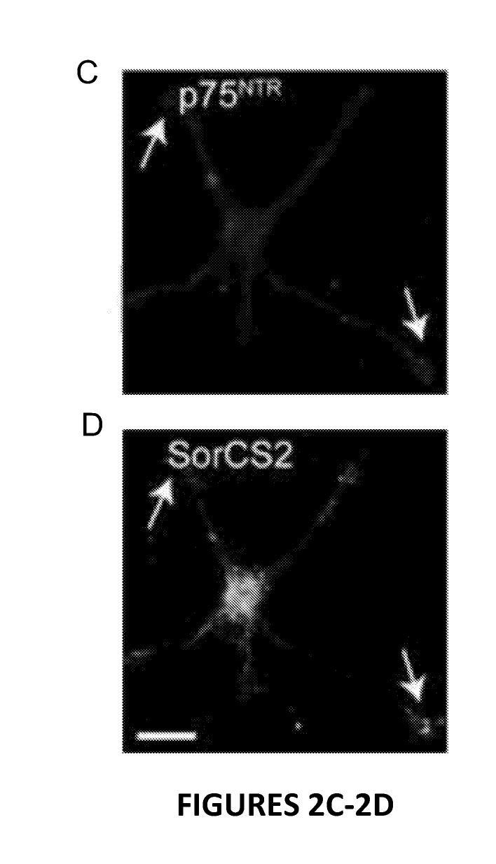

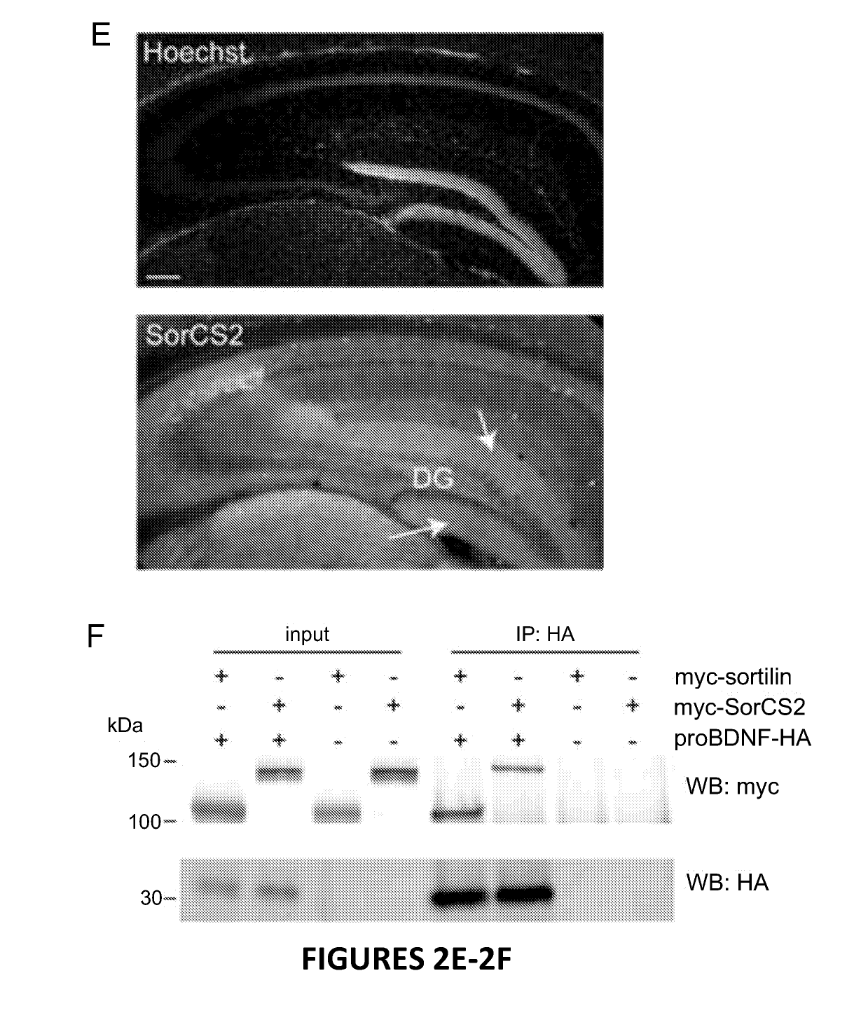

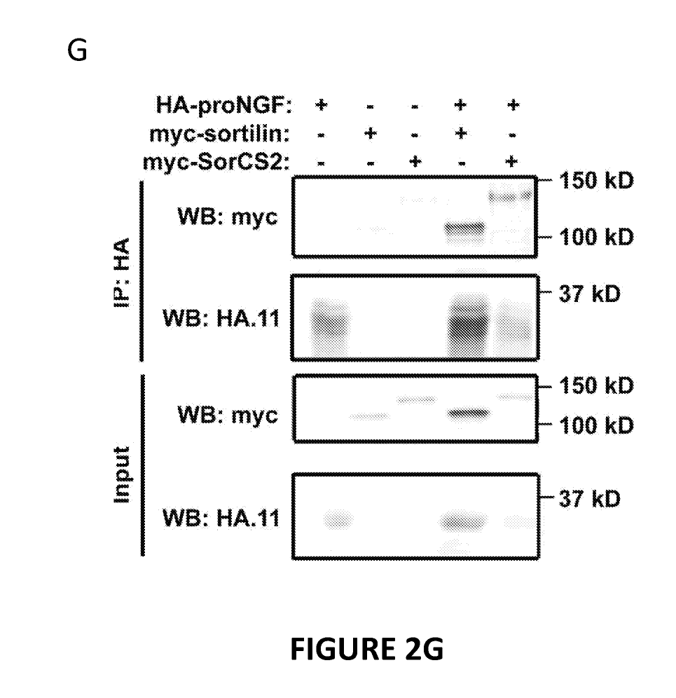

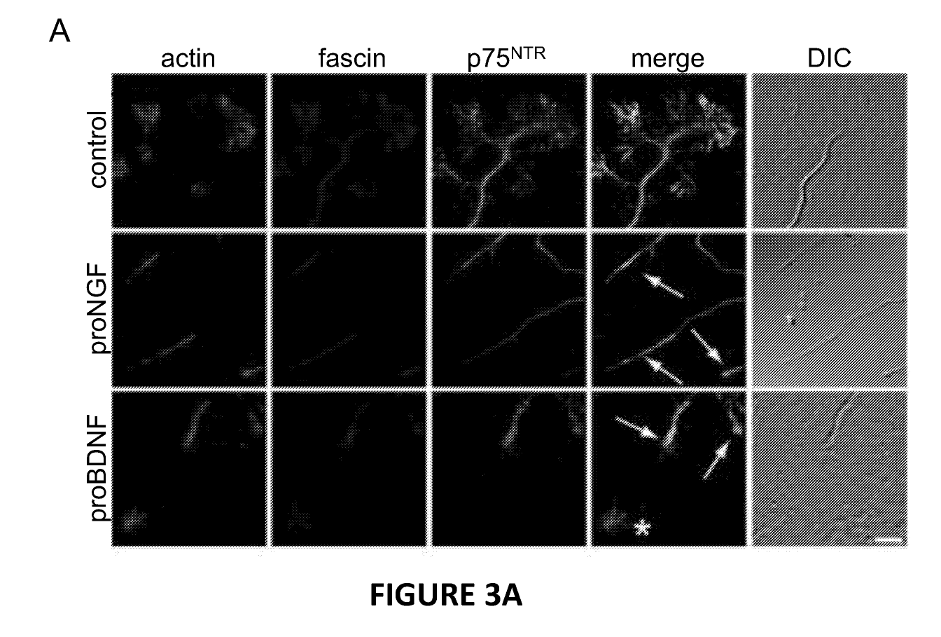

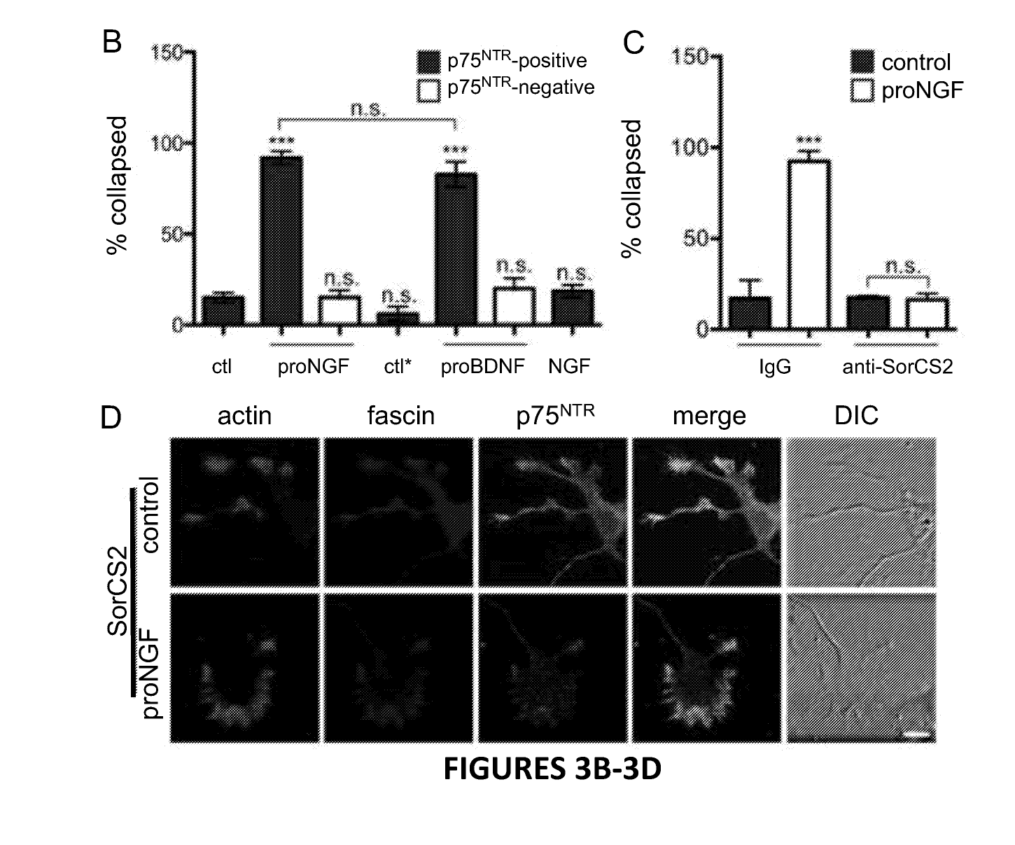

The inventors hypothesized that the morphological defects observed in the probdnf-HA/+ mouse might result from acute actions of proBDNF on neuronal cell shape, leading to reduced outgrowth during development. To test this hypothesis, the inventors transfected cultured mouse hippocampal neurons derived from E15.5 embryos at DIV2 with LifeAct-RFP, a short peptide linked to RFP that highlights filamentous actin (RIEDL, J. et al., Nat Methods 5:605-607 (2008)), and imaged living transfected cells on DIV3 before and after exposure to 0.1 nM proBDNF, or 0.1 nM proNGF. Since these hippocampal cultures express the BDNF receptor TrkB, but not the NGF receptor TrkA, the inventors performed all proBDNF experiments in presence of the Trk inhibitor K252a to rule out any interference of mature BDNF-TrkB signaling originating from the potential cleavage of proBDNF to mature BDNF. In the absence of the proNT, the movement of filopodia and growth cones was very dynamic, extending and retracting in different directions (FIGS. 2A and 2B, top panels). However, within 2 min after proNT addition, the motion of the growth cone froze and over the next few minutes, the now rigid actin structures collapsed (FIGS. 2A and 2B, bottom panels). This collapse was not limited to LifeAct-labeled filaments, as judged by DIC images and by staining for endogenous actin, fascin and p75^{NTR} (FIG. 3A). The effects of proNTs were observed in a subset of neurons, while other neurons in the same dish did not respond.

The observation that only some neurons responded to proNTs suggested that individual neurons expressed different receptors, leading to different responses to proNTs. Immunostaining of the E15.5 DIV3 hippocampal cultures revealed that about 25-30% of the neurons express p75^{NTR} (FIG. 2C). However, sortilin was not expressed by hippocampal neurons at this age, though it could be detected in cultures derived from older embryos (data not shown). In contrast, about 30-35% of the cells expressed SorCS2 (FIG. 2D), a member of the sortilin family of Vps10p domain-containing transmembrane receptors, which, just like sortilin, can interact with proBDNF (FIG. 2F). Most p75^{NTR} positive cells (˜97%) also expressed SorCS2, although some SorCS2 expressing cells were not p75^{NTR} positive (10-15%). Importantly, both p75^{NTR} and SorCS2 were also co-expressed in dentate granule cell dendrites of young mice (FIG. 2E, arrows), where the morphological defect in dendritic complexity was observed in vivo.

Therefore, the inventors assessed whether the population of cells responding to proNTs expressed p75^{NTR} by exposing cells to proNGF or proBDNF for 20 min, and then fixing and counterstaining for endogenous actin and the actin-bundling protein fascin to visualize cell protrusions, as well as for p75^{NTR}. Untreated cells display extended, fan-like growth cones and numerous filopodia, both rich in actin and fascin (FIG. 3A, top panel). Only cells expressing p75^{NTR} responded to proNT treatment with growth cone collapse and filopodial retraction (FIG. 3A, middle and bottom panels, see arrows; FIG. 3B). The inventors quantitated the response of growth cones from all neurites since, at this early stage in vitro, axonal and dendritic markers were not fully segregated. Growth cones of p75^{NTR} negative cells did not collapse after proNT addition (FIG. 3A, asterisk; FIG. 3B), nor did NGF lead to actin rearrangements in p75^{NTR} positive cells (FIG. 3B). Moreover, upon live imaging of LifeAct-RFP transfected neurons from NTR-/p75 mice, growth cone collapse was never observed following proNGF addition (not shown). Together, these results indicate that p75^{NTR} is required for proNT-mediated collapse of actin- and fascin-rich protrusions. As proBDNF and proNGF were comparable in inducing growth cone collapse (FIGS. 2A, B; 3A, B), and these hippocampal neurons express TrkB but not TrkA (not shown), proNGF was used for several of the in vitro studies to avoid interference with TrkB signaling stemming from potential processing of proBDNF to mature BDNF.

The p75^{NTR}-expressing hippocampal neurons in these cultures also expressed SorCS2, but not sortilin (FIG. 2D). To determine whether SorCS2 acted as a co-receptor with p75^{NTR} to induce retraction of actin-rich structures, the inventors added an anti-SorCS2 antibody that recognizes the SorCS2 ectodomain to these cultures for 20 min prior to addition of proNGF. Following 20 min exposure to proNGF, cells were fixed and counterstained for actin, fascin and p75^{NTR}. Pre-incubation with anti-SorCS2 antibody had no effect on neuronal morphology by itself, but efficiently blocked the ability of proNGF to induce filopodial collapse (FIG. 3C, D), while control IgG or anti-sortilin ectodomain antibodies did not impair the response to proNGF (FIG. 3C). Therefore, p75^{NTR} and SorCS2 act as co-receptors for proNGF that mediate acute alterations in actin morphology.

Proteomic Screen for Signaling Intermediates

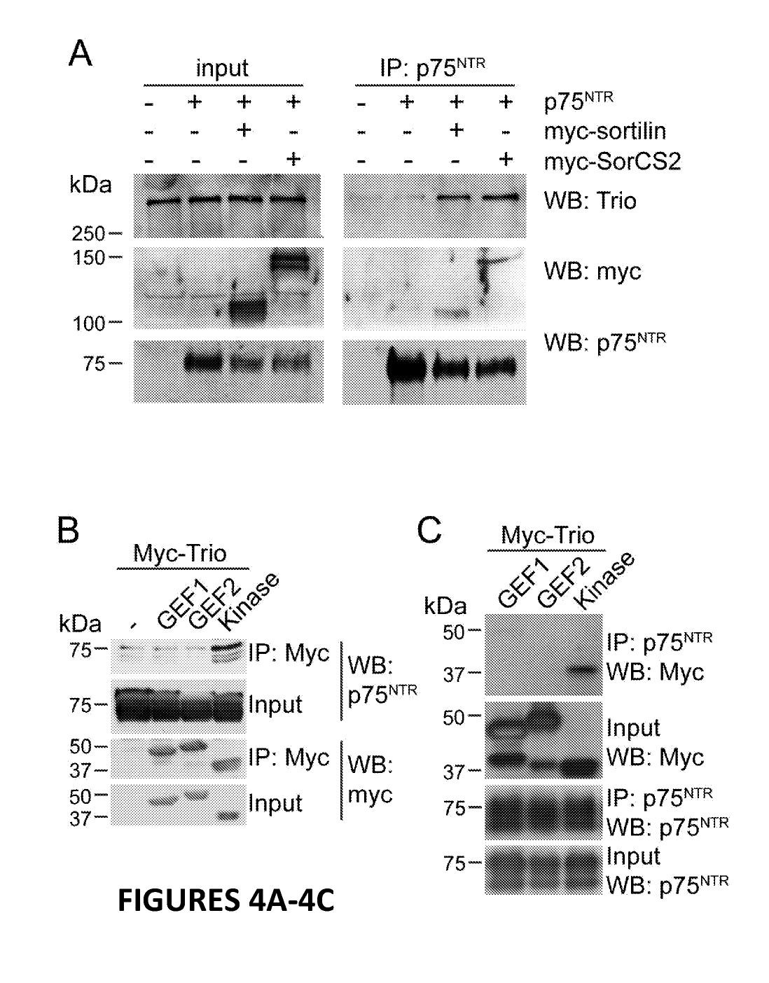

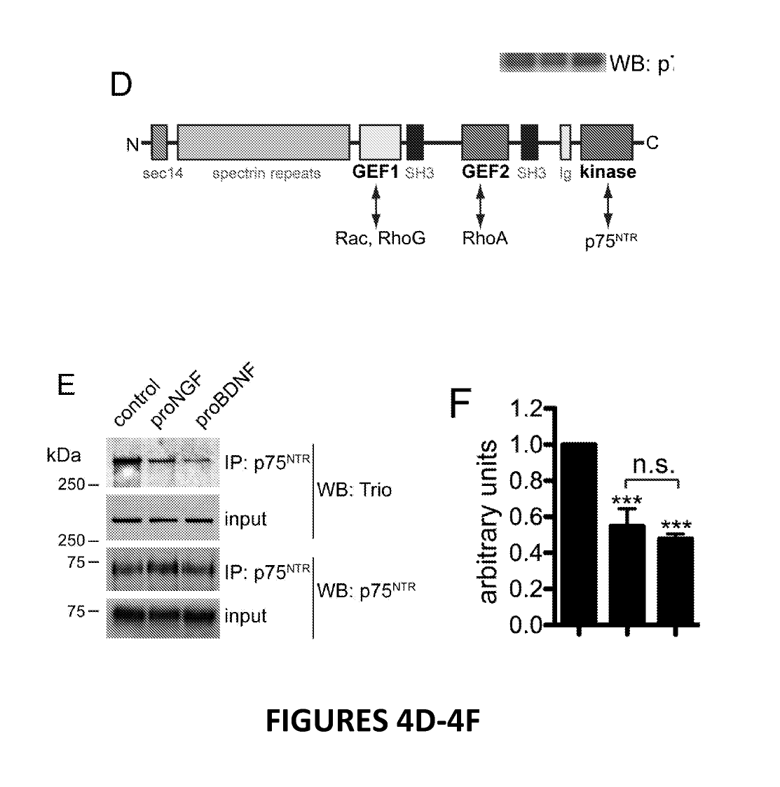

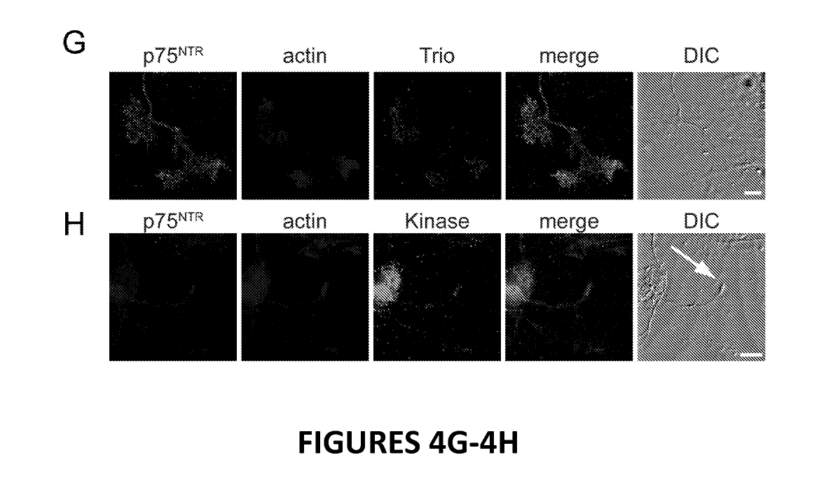

To identify molecules that lie downstream of the proNGF/p75^{NTR}/SorCS2 pathway in an unbiased manner, the inventors conducted a proteomic analysis of proteins that associated with p75^{NTR} and the SorCS2 family member sortilin. p75-interacting proteins were immunoprecipitated from HT1080 cells stably expressing p75 or p75 and sortilin, and those proteins that interacted with p75^{NTR} were identified in cells co-expressing both receptors, but not with p75^{NTR} alone. With this approach, the inventors identified the Rac and Rho GEF Trio, and confirmed the interaction by co-immunoprecipitation analysis. Indeed, co-expression of p75 and sortilin or p75 and SorCS2 led to the formation of a stable complex of p75^{NTR} with Trio (FIG. 4A), suggesting that SorCS2 acts similarly to sortilin in promoting an interaction between p75 and Trio. Using HEK293T cells, the inventors mapped the interaction of p75 to the kinase domain of Trio by co-precipitation analysis (FIG. 4B-D).

To assess whether ligand binding modulates the interaction of p75^{NTR} and Trio, the inventors expressed p75^{NTR} and SorCS2 in HT1080 cells, incubated the cells with proNGF or proBDNF for 20 min, and cell lysates were immunoprecipitated with anti-p75^{NTR}. Western blot analysis demonstrated that proNT addition markedly reduced the interaction between p75^{NTR}/SorCS2 and Trio (FIG. 4E, F), indicating that Trio was displaced from the p75^{NTR}/SorCS2 receptor complex upon proNT binding. To evaluate the role of Trio in primary E15 DIV3 hippocampal neurons, the inventors immuno-localized p75^{NTR}, actin and Trio. Consistent with a prior study (SEIPEL, K. et al., Journal of Cell Sci 112(Pt 12): 1825-1834 (1999)), endogenous Trio was observed to localize to actin-rich protrusions in these cells (FIG. 4G). ProNGF addition led a redistribution of Trio along the neuritis in p75^{NTR}-positive cells, while Trio remained enriched in intact growth cones in p75^{NTR}-negative cells. The inventors also confirmed the existence of the p75^{NTR}/SorCS2/Tio trimeric complex in embryonic rat brain lysates in vivo. Finally, the inventors assessed whether expression of the Trio kinase domain, or a kinase-dead mutant (trio kin^{K2921A}), would act in a dominant-negative manner to interfere with the interaction of p75^{NTR} with endogenous Trio, therefore displacing the Trio GEF1 and GEF2 activities. This would mimic proNT action and therefore should lead to collapse of actin structures even in absence of proNGF. As predicted, expression of the Trio kinase domain was sufficient to induce growth cone collapse in primary hippocampal neurons (FIG. 4H, arrow).

proNT Binding Leads to Rac Inactivation.

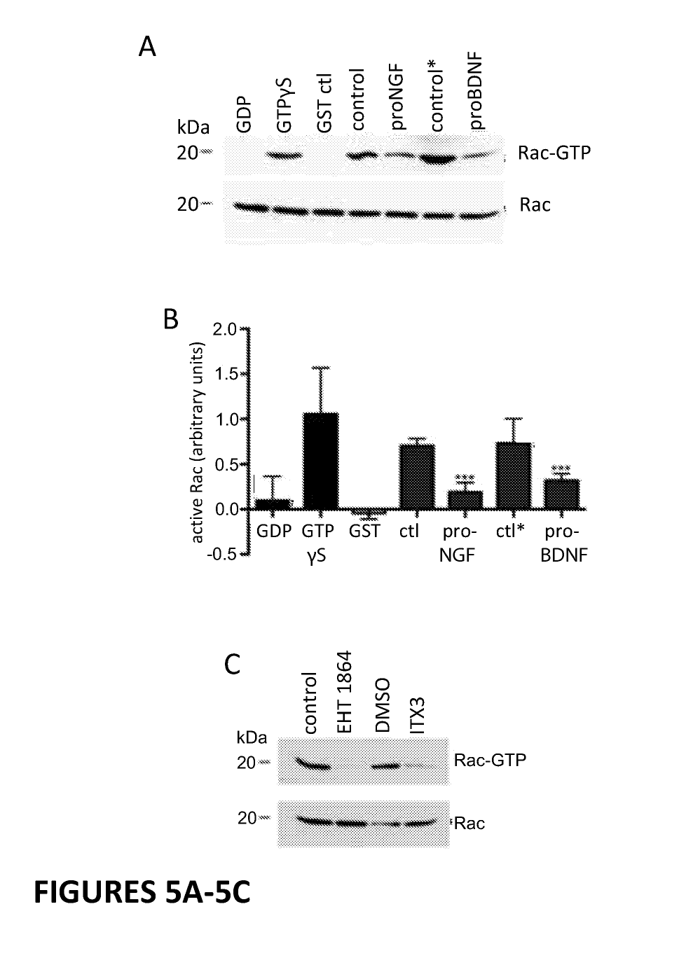

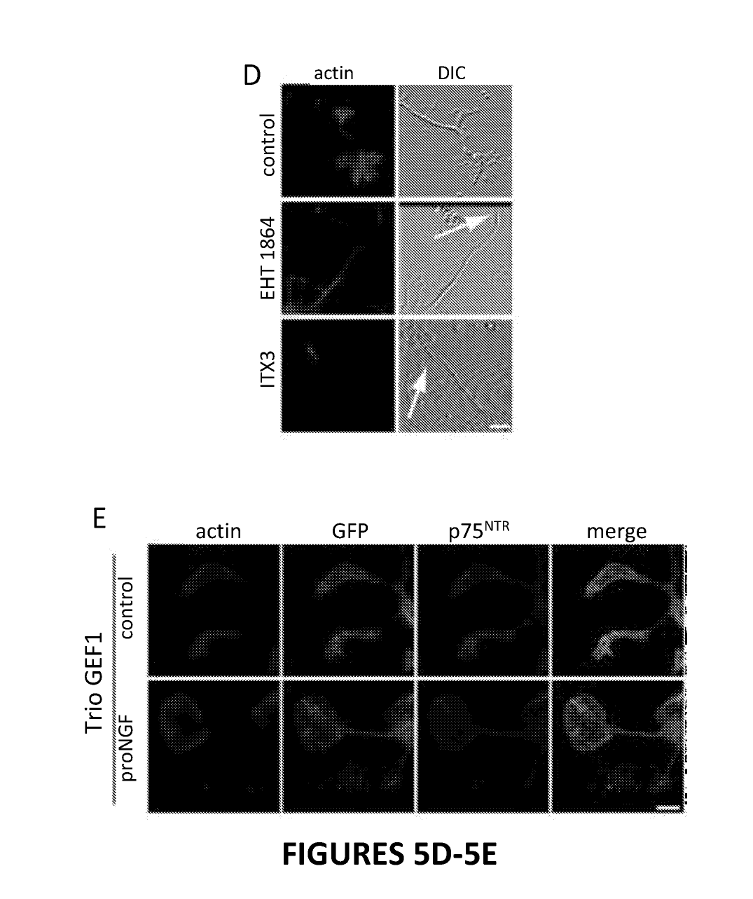

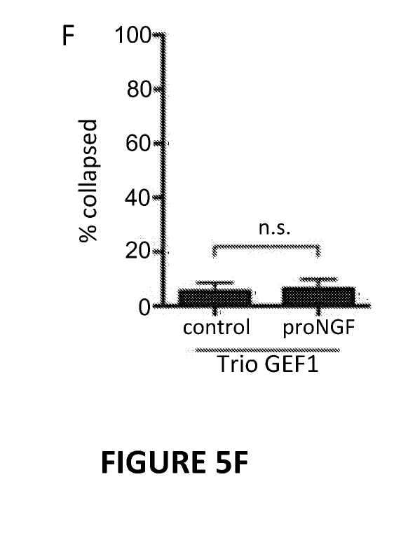

Since displacement of Trio from actin-rich structures was sufficient to induce growth cone collapse, the inventors assessed whether the localized action of the Rac- and RhoGactivating GEF1 domain, or RhoA activation by the GEF2 domain was required for growth cone integrity. Rac has been implicated in axon elongation and neurite outgrowth (KOZMA, R. et al., Mol Cell Biol 17:1201-1211 (1997); LUO, L., Nat Rev Neurosci 1:173-180 (2000)), as well as axon guidance and growth cone repulsion (JIN, Z. et al., Journal of Neuroscience 17:6256-6263 (1997)). Therefore, the inventors first assessed whether the observed collapse of actin structures was a consequence of Rac inactivation. For this, the inventors purified the Rac effector domain PAK-CRIB to isolate activated Rac (BENARD, V. et al., Methods Enzymol 345:349-359 (2002)) and assessed Rac activity in mouse E15 DIV2 cortical neurons before or after application of proNGF or proBDNF. DIV2 cortical neurons were used as a greater proportion of cells express p75^{NTR} at DIV 2 (approx. 50%) as compared to DIV3, and because of the higher yield of neurons from the cortex as compared to the hippocampus. Cortical neurons expressing p75^{NTR} also displayed growth cone collapse in response to proNGF (not shown). Quantification of activated Rac from untreated, proNGF- or proBDNF-treated cells demonstrated that proNT exposure led to a significant decrease in Rac activity (FIG. 5A, B). To determine whether inhibition of Rac could indeed cause growth cone collapse, hippocampal neurons were incubated with the specific Rac inhibitor EHT 1864, or the Trio GEF1 domain inhibitor ITX3 (BOUQUIER, N. et al., Chem Biol 16:657-666 (2009)). Both drugs caused a striking decrease in Rac activity in primary neuronal cultures (FIG. 5C), and led to the collapse of growth cone structures (FIG. 5D), confirming that Rac inactivation may underlie the retraction of actin-rich structures in response to proNTs. Finally, the inventors assessed whether overexpression of the GEF1 domain of Trio, which promotes Rac activation, could overcome the proNGF-induced inactivation of Rac and filopodial collapse. Indeed, it was found that exogenous expression of Trio GEF1 abolished proNGF induced filopodial retraction (FIG. 5E, F). In contrast, inactivation of RhoA using C3 transferase or the ROCK inhibitor Y-27632 neither induced growth cone collapse nor did they interferen with proNGF-mediated growth cone collapse. Taken together, these results suggest that proNT binding to the p75^{NTR}/SorCS2 complex leads to a displacement of Trio, reduced activation of Rac and subsequent collapse of actin-rich protrusions.

proNGF-Dependent Collapse Requires Fascin Phosphorylation.

Live imaging of neurons showed a biphasic collapse, in which a reduction in actin dynamics preceded the collapse of the then rigid structures. Therefore, the inventors examined a potential role for the actin filament stabilizing protein, fascin. The binding of fascin to elongating actin filaments has been reported to result in actin bundling and promotes the formation of stable filopodia (VIGNJEVIC, D. et al., Journal of Cell Biol 174:863-875 (2006)). Moreover, phosphorylation of serine 39 in fascin by protein kinase C (PKC) has been reported to lead to fascin inactivation and dissociation from actin filaments (ONO, S. et al., Journal of Biol Chem 272:2527-2533 (1997); YAMAKITA, Y. et al., Journal of Biol Chem 271:12632-12638 (1996)). It has also been reported that fascin can directly interact with p75^{NTR} (SHONUKAN, O. et al., Oncogene 22:3616-3623 (2003)). In addition, phosphorylation of fascin at serine 39 has previously been shown to abrogate proNGF dependent cell migration, assessed in non-neuronal cells (SHONUKAN, O. et al., Oncogene 22:3616-3623 (2003)) and dependent upon PKC activation. The inventors also found that fascin also interacted with p75^{NTR} in embryonic brain lysates. Thus, the inventors hypothesized that proNT-induced fascin phosphorylation and dissociation from actin filaments might contribute to both the reduction in actin dynamics, and to filament collapse.

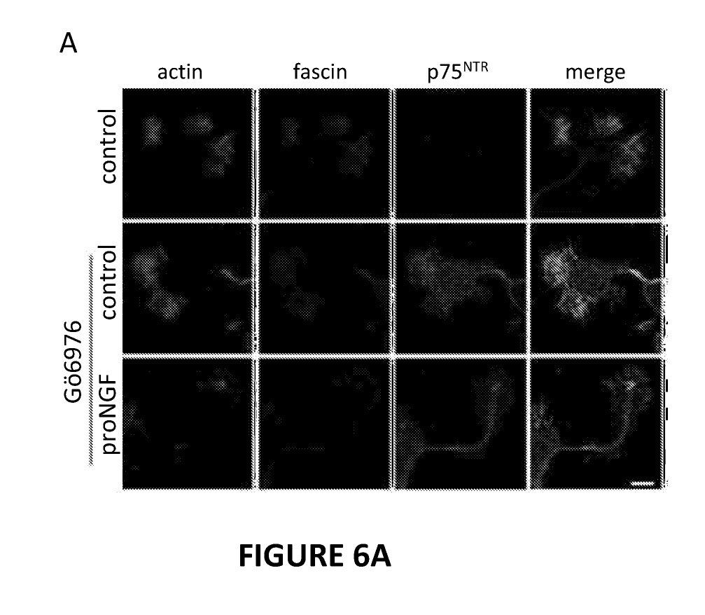

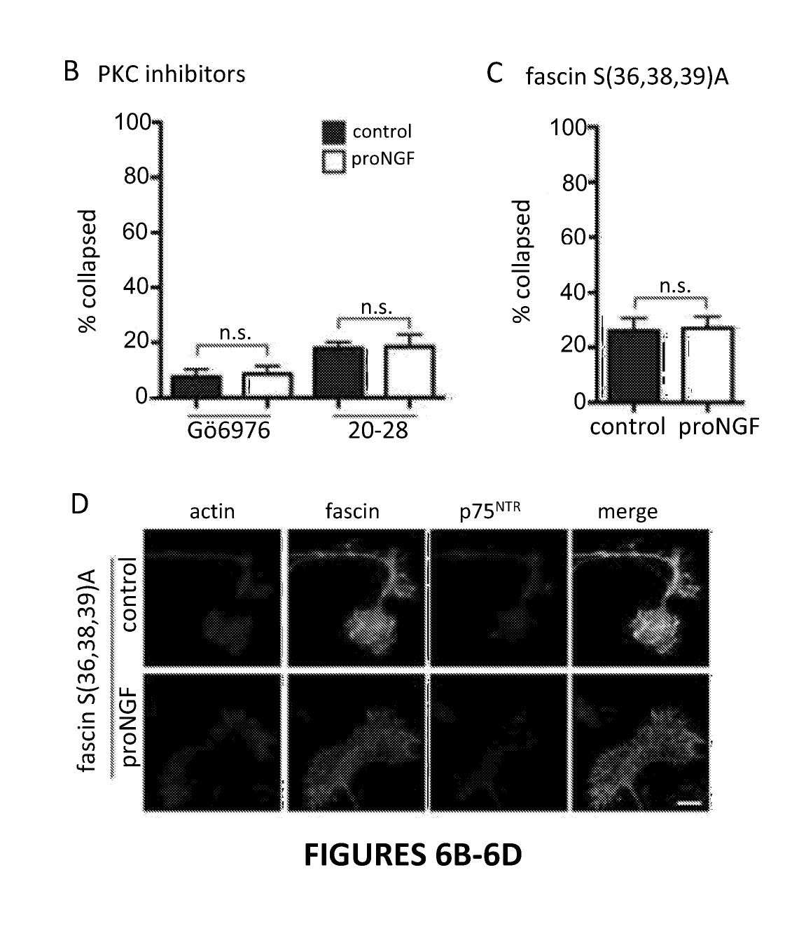

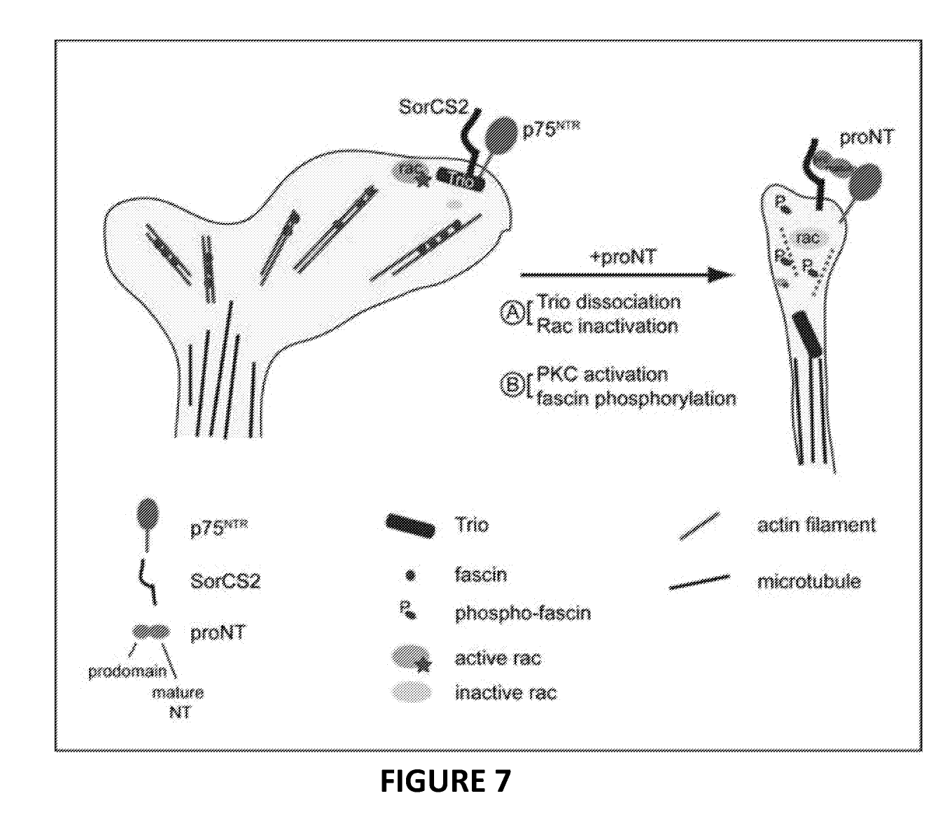

Therefore, the inventors first tested whether inhibition of PKC could prevent proNGF induced filopodial collapse. Hippocampal neurons (DIV3) were preincubated with the PKC inhibitor Gö6976 (FIG. 6A,B) or the small peptide inhibitor 20-28 (FIG. 6B) for 45 min prior to the addition of proNGF. In both cases, exposure to proNGF failed to induce collapse of actin-rich structures (FIG. 6A, B), suggesting that PKC-dependent phosphorylation was required for this process. To test if fascin is the PKC target, the non-phosphorylatable fascin mutant S(36,38,39)A was transfected into hippocampal neurons one day before proNGF addition. The serine-to-alanine mutations prevent PKC-dependent phosphorylation of fascin and mimic the active, actin-bundling form of the protein (VIGNJEVIC, D. et al., Journal of Cell Biol 174:863-875 (2006)). Cells expressing this mutant showed an altered morphology, with aberrant, less regularly aligned fascin bundles throughout the growth cones (FIG. 6D, top panel). These observations were similar to previous studies in melanoma cells (VIGNJEVIC, D. et al., Journal of Cell Biol 174:863-875 (2006)). However, addition of proNGF did not alter this morphology, with growth cones remaining extended (FIG. 6C, D, bottom panel), suggesting that fascin inactivation downstream of proNGF addition contributes to efficient growth cone collapse downstream of proNT action.

Altogether, these observations suggest that proNT signaling through the p75^{NTR}/SorCS2 complex leads to growth cone collapse through inactivation of Rac and fascin and subsequent retraction of actin-rich structures. During early postnatal development, when p75^{NTR}, SorCS2 and proBDNF are expressed in the dentate gyrus (YANG, J. et al., Nat Neuroscience 12:113-115 (2009)), proBDNF can negatively influence outgrowth and lead to reduced dendritic arborization in the dentate gyrus of proBDNF-HA/+ mice in vivo.

Summary of Experimental Results

First, the inventors have demonstrated new biological actions for proNTs in mediating acute remodeling of neuronal processes via p75^{NTR}. Importantly, these effects are mediated by proNTs, but not the mature neurotrophins, implying that the regulation of isoform conversion in turn controls acute morphological responses. These results indicate that the expression of proBDNF in postnatal development contributes to dendritic arborization, as exemplified by reduction in dendritic branching upon overexpression of proBDNF in vivo. Moreover, these studies indicate that proNGF, in addition to promoting apoptosis, may actively induce retraction of neuronal processes upon injury, following the rapid induction of p75^{NTR} and proNGF. Second, the inventors have identified a new sortilin family member, SorCS2 as a co-receptor with p75^{NTR} to mediate the effects of proNTs on growth cone collapse. Third, the inventors have identified two new signaling pathways downstream of p75^{NTR}/SorCS2 to actively reduce process outgrowth. ProNTs induce dissociation of Trio from a preformed complex with p75^{NTR}/SorCS2 to decrease active Rac. Concomitantly, binding of proNTs to p75^{NTR}/SorCS2 activates PKC to induce fascin phosphorylation and dissociation from actin filaments, to permit their rapid collapse in the growth cone.

What is claimed is:

1. A method of inhibiting neurite pruning or synaptic elimination in a subject, comprising administering to the subject an antagonist which inhibits the interaction of a proNT with p75^{NTR} and/or SorCS2.

2. The method of claim 1, wherein said proNT is elected from the group consisting of proNGF, proBDNF, proNT-3, and proNT-4/5.

3. The method of claim 1, wherein said antagonist is a proNT antagonist which inhibits the level or activity of said proNT.

4. The method of claim 3, wherein said proNT antagonist is an antibody specific for said proNT and inhibits the binding of the proNT to p75^{NTR} and/or SorCS2.

5. The method of claim 4, wherein said antibody is an antibody directed to the pro-domain of said proNT.

6. The method of claim 3, wherein said proNT antagonist is a nucleic acid or peptide aptamer which specifically binds to said proNT and inhibits the binding of said proNT to p75^{NTR} and/or SorCS2.

7. The method of claim 3, wherein said proNT antagonist is an oligopeptide or small molecule which inhibits the binding of said proNT to p75^{NTR} and/or SorCS2.

8. The method of claim 3, wherein said proNT antagonist is an anti-sense molecule or siRNA which reduces the level or activity of mRNA of said proNT.

9. The method of claim 1, wherein antagonist is a SorCS2 antagonist.

10. The method of claim 9, wherein said SorCS2 antagonist is an antibody directed to SorCS2 which blocks the binding of a proNT to SorCS2.

11. The method of claim 10, wherein said antibody is an antibody directed to the ectodomain domain of SorCS2.

12. The method of claim 9, wherein said SorCS2 antagonist is a nucleic acid or peptide aptamer which binds to SorCS2 and blocks the binding of a proNT to SorCS2.

13. The method of claim 9, wherein said SorCS2 antagonist is an oligopeptide or small molecule compound which inhibits the interaction of SorCS2 with a proNT and/or p75NTR.

14. The method of claim 9, wherein said SorCS2 antagonist is an anti-sense molecule or siRNA which reduces the level or activity of SorCS2 mRNA

15. The method of claim 1, wherein said subject suffers from an early stage or mild neurological disorder.

16. The method of claim 15, wherein said neurological disorder is a neurodegenerative disorder selected from Alzheimer's disease, Parkinson's disease, Huntington's disease, stroke, ALS, and peripheral neuropathies.

17. The method of claim 15, wherein said subject suffers from mild cognitive impairment.

18. The method of claim 1, wherein said antagonist is administered to the subject via ingestion, injection, or delivery to cerebral fluid via a needle or catheter.
